(19) United States
(12) Patent Application Publication
(10) Pub. No.: US 2026/0152762 A1
(43) Pub. Date: Jun. 4, 2026

(54) COMPOSITIONS AND METHODS FOR TREATING RETINITIS PIGMENTOSA

(71) Applicant: Biogen MA Inc., Cambridge, MA (US)

(72) Inventors: Kip Michael Connor, Lexington, MA (US); Edward Raymond Guilmette, Franklin, MA (US); Meihua Ju, Watertown, MA (US); Vsevolod Kostrubsky, Jacksonville, FL (US); Ashley Mackey, Somerville, MA (US); Devin Scott McDougald, Boston, MA (US); Junghae Suh Sparrow, Lexington, MA (US); Lindsay Lee Wong, Boston, MA (US)

(21) Appl. No.: 19/122,536

(22) PCT Filed: Oct. 20, 2023
(86) PCT No.: PCT/US23/35626

§ 371 Date: Apr. 18, 2025

Related U.S. Application Data
(60) Provisional application No. 63/418,263, filed on Oct. 21, 2022.

(51) Int. Cl. C12N 15/864; A61K 9/00; A61K 31/56; A61K 31/711; A61K 48/00; A61P 27/02; C12N 15/11

(52) U.S. Cl. CPC C12N 15/8645; A61K 9/0019; A61K 9/0048; A61K 31/56; A61K 31/711; A61K 48/005; A61P 27/02; C12N 15/11; C12N 2750/14143; C12N 2830/15; C12N 2830/34; C12N 2830/48; C12N 2830/50

(57) ABSTRACT

Among other things, the present disclosure provides compositions, e.g., isolated nucleic acids, vectors, and recombinant adeno-associated viruses (rAAVs), comprising a nucleic acid sequence encoding a PRPF31 polypeptide. In some embodiments, the present disclosure provides methods of increasing expression of PRPF31, e.g., in a subject. In some embodiments, the present disclosure provides methods of treating retinal degeneration in a subject. In some embodiments, the present disclosure provides methods of treating retinitis pigmentosa (RP) in a subject.

### CROSS-REFERENCE TO RELATED APPLICATIONS

This application claims priority to U.S. Provisional Application No. 63/418,263, filed on Oct. 21, 2022, the entirety of which is incorporated herein by reference.

### BACKGROUND

Retinitis pigmentosa (RP) is a degenerative genetic disease that affects cells in the retina. Retinitis pigmentosa is characterized by a decrease or loss of vision. Initial symptoms of retinitis pigmentosa have been reported to include impaired vision in low light settings, e.g., at night. Further progression may involve loss of peripheral vision followed by loss of central vision and subsequently, total blindness. Mutations in PRPF31 have been reported as having a causative role in some instances of retinal degeneration, e.g., retinitis pigmentosa. There are currently no approved treatments for retinitis pigmentosa associated with mutant PRPF31, and accordingly, there is a need for therapeutic modalities to treat PRPF31-associated retinal degeneration.

### SUMMARY

Among other things, the present disclosure provides technologies (e.g., one or more of isolated nucleic acids, vectors, recombinant adeno-associated viruses (rAAVs), compositions thereof, or methods thereof) for treating or preventing various diseases, disorders, or conditions. In some embodiments, a disease, disorder, or condition is associated with PRPF31. In some embodiments, a disease, disorder, or condition is associated with mutant PRPF31. In some embodiments, the present disclosure provides isolated nucleic acids, vectors, or rAAVs comprising a nucleic acid sequence encoding a PRPF31 polypeptide. In some embodiments, a PRPF31 polypeptide is a wild-type PRPF31 polypeptide. In some embodiments, a provided technology can increase the levels of PRPF31 transcript and/or polypeptide in a system or subject. In some embodiments, the present disclosure provides methods of increasing expression of PRPF31 in a system or subject. In some embodiments, the present disclosure provides methods of treating a subject susceptible to or suffering from a disease, disorder, or condition.

In some aspects, the present disclosure provides isolated nucleic acids. In some embodiments, an isolated nucleic acid comprises (i) an adeno-associated virus (AAV) 5′ inverted terminal repeat (ITR), (ii) a promoter, (iii) a nucleic acid sequence encoding a PRPF31 polypeptide, and (iv) an adeno-associated virus (AAV) 3′ inverted terminal repeat (ITR). In some embodiments, an isolated nucleic acid comprises (i) an adeno-associated virus (AAV) 5′ inverted terminal repeat (ITR), (ii) a promoter, (iii) a nucleic acid sequence encoding a PRPF31 polypeptide, (iv) a woodchuck hepatitis virus post-transcriptional regulatory element (WPRE), and (v) an adeno-associated virus (AAV) 3′ inverted terminal repeat (ITR).

In some embodiments, a PRPF31 polypeptide is a wild-type (WT) polypeptide. In some embodiments, a nucleic acid sequence described herein encodes a WT PRPF31 polypeptide. In some embodiments, a nucleic acid sequence encoding a PRPF31 polypeptide is or comprises the nucleic acid sequence of SEQ ID NO: 1.

In some embodiments, a promoter comprises a human cytomegalovirus (CMV) enhancer or portion thereof, a chicken beta-actin (CBA) promoter or portion thereof, a human ubiquitin C (UbC) enhancer or portion thereof, a splice donor, a splice acceptor, or a combination thereof. In some embodiments, a promoter comprises a human CMV enhancer or portion thereof. In some embodiments, a promoter comprises a CBA promoter or portion thereof. In some embodiments, a promoter comprises a human UbC enhancer or portion thereof. In some embodiments, a promoter comprises a splice donor. In some embodiments, a promoter comprises a splice acceptor. In some embodiments, a promoter comprises a splice donor and a splice acceptor. In some embodiments, a promoter is or comprises the nucleic acid sequence of SEQ ID NO: 4.

In some embodiments, a 5′ ITR is or comprises an AAV2 5′ ITR and/or a 3′ ITR is or comprises an AAV2 3′ ITR. In some embodiments, a 5′ ITR is or comprises an AAV2 5′ ITR. In some embodiments, a 3′ ITR is or comprises an AAV2 3′ ITR. In some embodiments, a 5′ ITR is or comprises the nucleic acid sequence of SEQ ID NO: 2. In some embodiments, a 3′ ITR is or comprises the nucleic acid sequence of SEQ ID NO: 3.

In some embodiments, a WPRE is or comprises the nucleic acid sequence of SEQ ID NO: 5.

In some embodiments, an isolated nucleic acid comprises a 3′ untranslated region (UTR) element. In some embodiments, a 3′ UTR element comprises a polyadenylation signal. In some embodiments, a polyadenylation signal comprises a human growth hormone (hGH) polyadenylation signal. In some embodiments, a polyadenylation signal comprises a beta-globin polyadenylation signal. In some embodiments, a polyadenylation signal comprises a human beta-globin polyadenylation signal. In some embodiments, a polyadenylation signal is or comprises the nucleic acid sequence of SEQ ID NO: 6.

In some embodiments, an isolated nucleic acid is or comprises the nucleic acid sequence of SEQ ID NO: 7.

In some aspects, the present disclosure provides vectors. In some embodiments, a vector comprises an isolated nucleic acid as described herein. In some embodiments, a vector is a viral vector. In some embodiments, a vector is a recombinant adeno-associated virus (rAAV).

In another aspect, the present disclosure provides recombinant adeno-associated viruses (rAAVs). In some embodiments, a rAAV comprises (i) a capsid and (ii) an isolated nucleic acid as described herein or a vector as described herein. In some embodiments, a rAAV comprises (i) a capsid and (ii) an isolated nucleic acid as described herein. In some embodiments, a rAAV comprises (i) a capsid and (ii) a vector as described herein. In some embodiments, a capsid is an AAV2 capsid.

In another aspect, the present disclosure provides pharmaceutical compositions. In some embodiments, a pharmaceutical composition comprises an isolated nucleic acid as described herein, a vector as described herein, or a rAAV as described herein. In some embodiments, a pharmaceutical composition comprises an isolated nucleic acid as described herein. In some embodiments, a pharmaceutical composition comprises a vector as described herein. In some embodiments, a pharmaceutical composition comprises a rAAV as described herein. In some embodiments, a pharmaceutical composition comprises (i) an isolated nucleic acid as described herein, a vector as described herein, or a rAAV as described herein and (ii) a pharmaceutically acceptable carrier or excipient. In some embodiments, a pharmaceutical composition comprises (i) an isolated nucleic as described herein and (ii) a pharmaceutically acceptable carrier or excipient. In some embodiments, a pharmaceutical composition comprises (i) a vector as described herein and (ii) a pharmaceutically acceptable carrier or excipient. In some embodiments, a pharmaceutical composition comprises (i) a rAAV as described herein and (ii) a pharmaceutically acceptable carrier or excipient.

In another aspect, the present disclosure provides cells. In some embodiments, a cell comprises an isolated nucleic acid as described herein, a vector as described herein, or a rAAV as described herein. In some embodiments, a cell comprises an isolated nucleic acid as described herein. In some embodiments, a cell comprises a vector as described herein. In some embodiments, a cell comprises a rAAV as described herein. In some embodiments, a cell is a host cell. In some embodiments, a cell is an isolated cell.

In another aspect, the present disclosure provides methods of increasing expression of PRPF31 in a subject. In some embodiments, a method of increasing expression of PRPF31 in a subject comprises administering to the subject an isolated nucleic acid as described herein, a vector as described herein, a rAAV as described herein, or a pharmaceutical composition as described herein. In some embodiments, a method of increasing expression of PRPF31 in a subject comprises administering to the subject an isolated nucleic acid as described herein. In some embodiments, a method of increasing expression of PRPF31 in a subject comprises administering to the subject a vector as described herein. In some embodiments, a method of increasing expression of PRPF31 in a subject comprises administering to the subject a rAAV as described herein. In some embodiments, a method of increasing expression of PRPF31 in a subject comprises administering to the subject a pharmaceutical composition as described herein. In some embodiments, a method of increasing expression of PRPF31 in a subject comprises administering to the subject a therapeutically effective amount of an isolated nucleic acid as described herein, a vector as described herein, a rAAV as described herein, or a pharmaceutical composition as described herein. In some embodiments, a method of increasing expression of PRPF31 in a subject comprises administering to the subject a therapeutically effective amount of an isolated nucleic acid as described herein. In some embodiments, a method of increasing expression of PRPF31 in a subject comprises administering to the subject a therapeutically effective amount of a vector as described herein. In some embodiments, a method of increasing expression of PRPF31 in a subject comprises administering to the subject a therapeutically effective amount of a rAAV as described herein. In some embodiments, a method of increasing expression of PRPF31 in a subject comprises administering to the subject a therapeutically effective amount of a pharmaceutical composition as described herein.

In some embodiments, a subject is susceptible to or suffering from a disease, disorder, or condition. In some embodiments, a disease, disorder, or condition is retinal degeneration. In some embodiments, a disease, disorder, or condition is retinitis pigmentosa. In some embodiments, a disease, disorder, or condition is retinitis pigmentosa-11 (RP11). In some embodiments, a subject has a mutation in one or both PRPF31 genes.

In some embodiments, a subject is an animal. In some embodiments, a subject is a mammal. In some embodiments, a subject is a mouse. In some embodiments, a subject is a rat. In some embodiments, a subject is a non-human primate. In some embodiments, a subject is a monkey. In some embodiments, a subject is a human.

In some embodiments, an isolated nucleic acid, vector, rAAV, or pharmaceutical composition is administered by injection. In some embodiments, an isolated nucleic acid is administered by injection. In some embodiments, a vector is administered by injection. In some embodiments, a rAAV is administered by injection. In some embodiments, a pharmaceutical composition is administered by injection. In some embodiments, an isolated nucleic acid, vector, rAAV, or pharmaceutical composition is administered by subretinal injection. In some embodiments, an isolated nucleic acid is administered by subretinal injection. In some embodiments, a vector is administered by subretinal injection. In some embodiments, a rAAV is administered by subretinal injection. In some embodiments, a pharmaceutical composition is administered by subretinal injection.

In some embodiments, a vector, rAAV, or pharmaceutical composition is administered at a dose within a range of about 10⁸ to about 10¹⁴ vector genome copies (vg) per eve. In some embodiments, a vector, rAAV, or pharmaceutical composition is administered at a dose of about 108, about 109, about 10¹⁰, about 10¹¹, about 10¹², about 10¹³, or about 10¹⁴ vector genome copies (vg) per eye. In some embodiments, a vector, rAAV, or pharmaceutical composition is administered at a dose of about 10⁸ vg per eye. In some embodiments, a vector, rAAV, or pharmaceutical composition is administered at a dose of about 5×10⁸ vg per eye. In some embodiments, a vector, rAAV, or pharmaceutical composition is administered at a dose of about 10⁹ vg per eve. In some embodiments, a vector, rAAV, or pharmaceutical composition is administered at a dose of about 5×10⁹ vg per eye. In some embodiments, a vector, rAAV, or pharmaceutical composition is administered at a dose of about 10¹⁰ vg per eye. In some embodiments, a vector, rAAV, or pharmaceutical composition is administered at a dose of about 5×10¹⁰ vg per eye. In some embodiments, a vector, rAAV, or pharmaceutical composition is administered at a dose of about 10¹¹ vg per eye. In some embodiments, a vector, rAAV, or pharmaceutical composition is administered at a dose of about 5×10¹¹ vg per eye. In some embodiments, a vector, rAAV, or pharmaceutical composition is administered at a dose of about 10¹² vg per eye. In some embodiments, a vector, rAAV, or pharmaceutical composition is administered at a dose of about 5×10¹² vg per eye.

In some embodiments, a method of increasing expression of PRPF31 in a subject further comprises administering to the subject one or more immunosuppressants. In some embodiments, one or more immunosuppressants are administered before, concurrently to, and/or following administration of an isolated nucleic acid, vector, rAAV, or pharmaceutical composition. In some embodiments, one or more immunosuppressants are administered before administration of an isolated nucleic acid, vector, rAAV, or pharmaceutical composition. In some embodiments, one or more immunosuppressants are administered concurrently to administration of an isolated nucleic acid, vector, rAAV, or pharmaceutical composition. In some embodiments, one or more immunosuppressants are administered following administration of an isolated nucleic acid, vector, rAAV, or pharmaceutical composition. In some embodiments, one or more immunosuppressants comprises a steroid. In some embodiments, one or more immunosuppressants comprises methylprednisolone.

In some aspects, the present disclosure provides methods of treating a subject susceptible to or suffering from a disease, disorder, or condition. In some embodiments, a method of treating a subject susceptible to or suffering from a disease, disorder, or condition comprises administering to the subject an isolated nucleic acid as described herein, a vector as described herein, a rAAV as described herein, or a pharmaceutical composition as described herein. In some embodiments, a method of treating a subject susceptible to or suffering from a disease, disorder, or condition comprises administering to the subject an isolated nucleic acid as described herein. In some embodiments, a method of treating a subject susceptible to or suffering from a disease, disorder, or condition comprises administering to the subject a vector as described herein. In some embodiments, a method of treating a subject susceptible to or suffering from a disease, disorder, or condition comprises administering to the subject a rAAV as described herein. In some embodiments, a method of treating a subject susceptible to or suffering from a disease, disorder, or condition comprises administering to the subject a pharmaceutical composition as described herein. In some embodiments, a method of treating a subject susceptible to or suffering from a disease, disorder, or condition comprises administering to the subject a therapeutically effective amount of an isolated nucleic acid as described herein, a vector as described herein, a rAAV as described herein, or a pharmaceutical composition as described herein. In some embodiments, a method of treating a subject susceptible to or suffering from a disease, disorder, or condition comprises administering to the subject a therapeutically effective amount of an isolated nucleic acid as described herein. In some embodiments, a method of treating a subject susceptible to or suffering from a disease, disorder, or condition comprises administering to the subject a therapeutically effective amount of a vector as described herein. In some embodiments, a method of treating a subject susceptible to or suffering from a disease, disorder, or condition comprises administering to the subject a therapeutically effective amount of a rAAV as described herein. In some embodiments, a method of treating a subject susceptible to or suffering from a disease, disorder, or condition comprises administering to the subject a therapeutically effective amount of a pharmaceutical composition as described herein.

In some embodiments, a subject is susceptible to or suffering from a disease, disorder, or condition. In some embodiments, a disease, disorder, or condition is retinal degeneration. In some embodiments, a disease, disorder, or condition is retinitis pigmentosa. In some embodiments, a disease, disorder, or condition is retinitis pigmentosa-11 (RP11). In some embodiments, a subject has a mutation in one or both PRPF31 genes.

In some embodiments, a subject is an animal. In some embodiments, a subject is a mammal. In some embodiments, a subject is a non-human primate. In some embodiments, a subject is a human.

In some embodiments, an isolated nucleic acid, vector, rAAV, or pharmaceutical composition is administered by injection. In some embodiments, an isolated nucleic acid is administered by injection. In some embodiments, a vector is administered by injection. In some embodiments, a rAAV is administered by injection. In some embodiments, a pharmaceutical composition is administered by injection. In some embodiments, an isolated nucleic acid, vector, rAAV, or pharmaceutical composition is administered by subretinal injection. In some embodiments, an isolated nucleic acid is administered by subretinal injection. In some embodiments, a vector is administered by subretinal injection. In some embodiments, a rAAV is administered by subretinal injection. In some embodiments, a pharmaceutical composition is administered by subretinal injection.

In some embodiments, a vector, rAAV, or pharmaceutical composition is administered at a dose within a range of about 10⁸ to about 10¹⁴ vector genome copies (vg) per eye. In some embodiments, a vector, rAAV, or pharmaceutical composition is administered at a dose of about 10⁸, about 10⁹, about 10¹⁰, about 10¹¹, about 10¹², about 10¹³, or about 10¹⁴ vector genome copies (vg) per eye. In some embodiments, a vector, rAAV, or pharmaceutical composition is administered at a dose of about 10⁸ g per eye. In some embodiments, a vector, rAAV, or pharmaceutical composition is administered at a dose of about 5×10⁸ vg per eye. In some embodiments, a vector, rAAV, or pharmaceutical composition is administered at a dose of about 10⁹ vg per eye. In some embodiments, a vector, rAAV, or pharmaceutical composition is administered at a dose of about 5×10⁹ vg per eye. In some embodiments, a vector, rAAV, or pharmaceutical composition is administered at a dose of about 10¹⁰ vg per eye. In some embodiments, a vector, rAAV, or pharmaceutical composition is administered at a dose of about 5×10¹⁰ vg per eye. In some embodiments, a vector, rAAV, or pharmaceutical composition is administered at a dose of about 10¹¹ vg per eye. In some embodiments, a vector, rAAV, or pharmaceutical composition is administered at a dose of about 5×10¹¹ vg per eye. In some embodiments, a vector, rAAV, or pharmaceutical composition is administered at a dose of about 10¹² vg per eve. In some embodiments, a vector, rAAV, or pharmaceutical composition is administered at a dose of about 5×10¹² vg per eye.

In some embodiments, a method of increasing expression of PRPF31 in a subject further comprises administering to the subject one or more immunosuppressants. In some embodiments, one or more immunosuppressants are administered before, concurrently to, and/or following administration of an isolated nucleic acid, vector, rAAV, or pharmaceutical composition. In some embodiments, one or more immunosuppressants are administered before administration of an isolated nucleic acid, vector, rAAV, or pharmaceutical composition. In some embodiments, one or more immunosuppressants are administered concurrently to administration of an isolated nucleic acid, vector, rAAV, or pharmaceutical composition. In some embodiments, one or more immunosuppressants are administered following administration of an isolated nucleic acid, vector, rAAV, or pharmaceutical composition. In some embodiments, one or more immunosuppressants comprises a steroid.

### BRIEF DESCRIPTION OF THE DRAWINGS

FIG. 1. Exemplary schematic of an isolated nucleic acid as described herein. From left-to-right, the diagram displays a 5′ inverted terminal repeat (ITR), a human cytomegalovirus enhancer (CMV enh.), a chicken beta-actin promoter (CβA prom.), a human ubiquitin C enhancer (UbC enh.), native human PRPF31 cDNA, a woodchuck hepatitis virus post-transcriptional regulatory element (WPRE), a polyadenylation signal (polyA), and a 3′ inverted terminal repeat (ITR).

FIG. 2. Exemplary schematic of subretinal administration. As depicted, a composition as described herein may be administered to an eye of a subject by subretinal injection. A subretinal bleb may be formed by subretinal injection of a composition.

FIG. 3A. Exemplary results from an in vivo assay of a rAAV as described herein. Cynomolgus macaques received subretinal injections as depicted in FIG. 2 of a rAAV comprising the construct depicted in FIG. 1. The PRPF31 polypeptide expressed from the rAAV comprised a V5 epitope tag fused to the N-terminus. At 28 days following administration, animals were euthanized and eyes were harvested, fixed, and processed as retinal cross sections for histological analysis as known in the art. Retinal cross sections were immunolabeled with antibodies against the V5 epitope tag (to identify vector-borne PRPF31) and DAPI. Representative image of one such retinal cross section is shown. RPE=retinal pigment epithelium; IS/OS=inner and outer segments; ONL=outer nuclear layer; INL=inner nuclear layer; GCL=ganglion cell layer.

FIG. 3B. Exemplary results from an in vivo assay of a rAAV as described herein. Cynomolgus macaques received subretinal injections as depicted in FIG. 2 of a rAAV comprising the construct depicted in FIG. 1. The PRPF31 polypeptide expressed from the rAAV comprised a V5 epitope tag fused to the N-terminus. Animals were treated with methylprednisolone on the day before administration of the rAAV and once per week thereafter. Inflammation was assessed on day 12 before administration and days 3, 7, 14, 21, and 28 following administration. Shown are representative results from one animal. The X axis depicts the days post-injection, while the Y axis depicts the vitreous cell scoring (0=none, 1=minimal, 2=mild, 3=moderate, 4=marked, 5=severe).

FIG. 4. Exemplary results from an in vivo assay of a rAAV as described herein. Mutant (Prpf31^{+/-}) mice received subretinal injections of a rAAV comprising the construct depicted in FIG. 1 (AAV-PRPF31) or a vehicle control. The PRPF31 polypeptide expressed from the rAAV comprised a V5 epitope tag fused to the N-terminus. Wild-type (Prpf31^{+/+}) mice which did not receive an injection were also examined. At 13 weeks post-injection, animals were sacrificed and retinal tissues were collected to examine vector-borne PRPF31 expression. For mutant (Prpf31^{+/-}) mice which received AAV-PRPF31, retinal tissue from within the subretinal bleb area (as formed by the subretinal injection as depicted in FIG. 2) and from outside the subretinal bleb area were examined. Retinal cross-sections were immunolabeled with antibodies against PRPF31 (top row of micrographs) and V5 (second row of micrographs), and DAPI (shown in the third row alongside a merge of the top two rows).

FIG. 5A. Exemplary results from an in vivo assay of a rAAV as described herein. Mutant (Prpf31^{+/-}) mice received subretinal injections of 2×10⁹ vg/eye of a rAAV comprising the construct depicted in FIG. 1 (N=5) or a vehicle control (N=4). At 13 weeks following injection, animals were sacrificed and eyes were harvested. Posterior eye cups (PECs) containing the (RPE) layer were dissociated from the neural retina and underwent protein extraction. Protein content was analyzed by western blot. An approximate 54 kDa band associated with PRPF31 was observed (top) and expression of PRPF31 was quantified and normalized to the corresponding expression of GAPDH (middle). As shown in the graph at bottom, a significant increase (>2 fold difference) in PRPF31 expression was observed in samples from mice that were injected with the rAAV comprising the PRPF31 cDNA as compared to mice that received the vehicle control. Error bars represent standard error of the mean (SEM). Unpaired Student's t-test; P=0.0006.

FIG. 5B. Exemplary results from an in vivo assay of a rAAV as described herein. Mutant (Prpf31^{+/-}) mice received subretinal injections of 2×10¹⁰ vg/eye of a rAAV comprising the construct depicted in FIG. 1 (N=5) or a vehicle control (N=4). At 13 weeks following injection, animals were sacrificed and eyes were harvested. Posterior eye cups (PECs) containing the (RPE) layer were dissociated from the neural retina and underwent protein extraction. Protein content was analyzed by western blot. An approximate 54 kDa band associated with PRPF31 was observed (top) and expression of PRPF31 was quantified and normalized to the corresponding expression of R-actin (middle). As shown in the graph at bottom, a significant increase (approximately a 4 fold difference) in PRPF31 expression was observed in samples from mice that were injected with the rAAV comprising PRPF31 cDNA as compared to mice that received the vehicle control. Error bars represent standard error of the mean (SEM). Unpaired Student's t-test; P<0.0001.

FIG. 6A. Exemplary results from an in vivo assay of a rAAV as described herein. Mutant (Prpf31^{+/-}) mice received subretinal injections of a vehicle control (N=6). Wild-type (Prpf31^{+/+}) mice which did not receive an injection were also examined (N=4). At 13 weeks post-injection, animals were sacrificed and retinal tissues were collected to examine RPE phagocytosis. Retinal cross-sections were immunolabeled with antibodies against PRPF31 (top row of micrographs), F-actin (second row of micrographs), and rhodopsin (RHO) (third row of micrographs) and DAPI (shown in the fourth row alongside a merge of the top three rows).

FIG. 6B. Exemplary results from an in vivo assay of a rAAV as described herein. Mutant (Prpf31^{+/-}) mice received subretinal injections of 2×10⁹ (Low Dose: N=5) or 2×10¹⁰ vg/eye (High Dose; N=4). At 13 weeks post-injection, animals were sacrificed and retinal tissues were collected to examine RPE phagocytosis. Retinal cross-sections were immunolabeled with antibodies against PRPF31 (top row of micrographs), F-actin (second row of micrographs), and rhodopsin (RHO) (third row of micrographs) and DAPI (shown in the fourth row alongside a merge of the top three rows).

FIG. 6C. Exemplary results from an in vivo assay of a rAAV as described herein. Mutant (Prpf31^{+/-}) mice received subretinal injections of 2×10⁹ (Low Dose: N=5) or 2×10¹⁰ vg/eye (High Dose; N=4) of a rAAV comprising the construct depicted in FIG. 1 or a vehicle control (N=6). Wild-type (Prpf31^{+/+}) mice which did not receive an injection were also examined (N=4). At 13 weeks post-injection, animals were sacrificed and retinal tissues were collected to examine RPE phagocytosis. Retinal cross-sections were immunolabeled with antibodies against rhodopsin (RHO) and PRPF31. RHO phagosomes in the RPE layer were quantified per 100 μm sections. Unpaired Student's t-test; for wild-type vs. vehicle, P=0.02; for wild-type vs. high dose, not significant (n.s.); for vehicle vs. low dose, P=0.01, for vehicle vs. high dose, P=0.0042. Error bars represent standard error of the mean (SEM).

FIG. 7. Exemplary results from an in vitro assay of a rAAV as described herein. Wild-type (Prpf31^{+/+}) and mutant (Prpf31^{+/-}) induced pluripotent stem cell (iPSC)-derived RPE cells were plated and matured for three weeks. A rAAV comprising the construct depicted in FIG. 1 were administered to the cultures at a MOI of 5×10⁵. Cells were cultured for three weeks post-administration before phagocytic capacity was assayed by treatment with FITC-conjugated photoreceptor outer segments (POS). Cells were evaluated thereafter by microscopy. Mutant (Prpf31^{+/-}) cells display a reduced ability to bind and internalize POS as compared to wild-type (Prpf31^{+/+}) cells as shown in the decreased FITC signal in the untreated column of micrographs. Treatment of cells with the rAAV comprising PRPF31 cDNA restored phagocytosis to the cells as demonstrated by the increased FITC signal following binding and internalization of POS (rightmost column of micrographs).

### DEFINITIONS

As used herein in the present disclosure, unless otherwise clear from context, (i) the term “a” or “an” may be understood to mean “at least one”; (ii) the term “or” may be understood to mean “and/or”: (iii) the terms “comprising”, “comprise”, “including” (whether used with “not limited to” or not), and “include” (whether used with “not limited to” or not) may be understood to encompass itemized components or steps whether presented by themselves or together with one or more additional components or steps: (iv) the term “another” may be understood to mean at least an additional/second one or more; and (v) where ranges are provided, endpoints are included.

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%. 2%, 1%, or less of the referred value.

Adeno-associated virus (AAV): As used herein, the terms “Adeno-associated virus” and “AAV” refer to viral particles, in whole or in part, of family Parvoviridae and genus Dependoparvovirus. AAV is a small, replication-defective, non-enveloped virus. AAV may include, but is not limited to, AAV serotype 1, AAV serotype 2, AAV serotype 3 (including serotypes 3A and 3B), AAV serotype 4, AAV serotype 5, AAV serotype 6, AAV serotype 7, AAV serotype 8, AAV serotype 9, AAV serotype 10, AAV serotype 11, AAV serotype 12, AAV serotype 13, AAV serotype rh10, AAV serotype rh74, AAV from the HSC 1-17 series, AAV from the CBr, CLv or CLg series, snake AAV, avian AAV, bovine AAV, canine AAV, equine AAV, ovine AAV, goat AAV, shrimp AAV, and any variant of any of the foregoing. AAV may also include engineered or chimeric versions of a wild-type AAV that include one or more insertions, deletions and/or substitutions within the Cap polypeptide(s) that affect one or more properties of the wild-type AAV serotype, including without limitation tropism and evasion of neutralizing antibodies (e.g., AAV-DJ, AAV-PHP.B, AAV-PHP.N, AAV.CAP-B1 to AAV.CAP-B25 and variants thereof). Wild-type AAV is replication deficient and requires co-infection of cells by a helper virus (e.g., adenovirus, herpes, or vaccinia virus) or supplementation of helper viral genes in order to replicate.

Administration: As used herein, the term “administration” refers to the administration of a composition to a subject. Administration may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, subretinal, topical, tracheal (including by intratracheal instillation), transdermal, vaginal, vitreal, or any combination thereof. In some embodiments, administration may be subretinal. In some embodiments, a preferred method of administration will reduce or prevent an immune response from a subject receiving treatment.

Agent: The term “agent” as used herein may refer to a compound or entity of any chemical class including, for example, polypeptides, nucleic acids, saccharides, lipids, small molecules, metals, or combinations thereof. As will be clear from context, in some embodiments, an agent can be or comprise a cell or organism, or a fraction, extract, or component thereof. In some embodiments, an agent is agent is or comprises a natural product in that it is found in and/or is obtained from nature. In some embodiments, an agent is or comprises one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents are provided as collections or libraries, for example that may be screened to identify or characterize active agents within them. Some particular embodiments of agents that may be utilized in accordance with the present disclosure include small molecules, antibodies, antibody fragments, aptamers, siRNAs, shRNAs, miRNAs, DNA/RNA hybrids, antisense oligonucleotides, ribozymes, peptides, peptide mimetics, small molecules, etc. In some embodiments, an agent is or comprises a polymer. In some embodiments, an agent is not a polymer and/or is substantially free of any polymer. In some embodiments, an agent contains at least one polymeric moiety. In some embodiments, an agent lacks or is substantially free of any polymeric moiety.

Complementary: As used herein, the term “complementary” in the context of nucleic acid base-pairing refers to oligonucleotide hybridization related by base-pairing rules. For example, the sequence “C-A-G-T” is complementary to the sequence “G-T-C-A.” Complementarity can be partial or total. Thus, any degree of partial complementarity is intended to be included within the scope of the term “complementary” provided that the partial complementarity permits oligonucleotide hybridization. Partial complementarity is where one or more nucleic acid bases is not matched according to the base pairing rules. Total or complete complementarity between nucleic acids is where each and every nucleic acid base is matched with another base under the base pairing rules.

Therapeutically effective amount: As used herein, the term “therapeutically effective amount” means an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. It is specifically understood that particular subjects may, in fact, be “refractory” to a “therapeutically effective amount.” To give but one example, a refractory subject may have a low bioavailability such that clinical efficacy is not obtainable. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in some embodiments, a therapeutically effective amount may be formulated and/or administered in a single dose. In some embodiments, a therapeutically effective amount may be formulated and/or administered in a plurality of doses, for example, as part of a dosing regimen.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. As will be understood by those skilled in the art, a variety of algorithms are available that permit comparison of sequences in order to determine their degree of homology, including by permitting gaps of designated length in one sequence relative to another when considering which residues “correspond” to one another in different sequences. Calculation of the percent identity between two nucleic acid sequences, for example, can be performed by aligning the two sequences for optimal comparison purposes (e.g., gaps can be introduced in one or both of a first and a second nucleic acid sequences for optimal alignment and non-corresponding sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. Representative algorithms and computer programs useful in determining the percent identity between two nucleotide sequences include, for example, the algorithm of Meyers and Miller (CABIOS, 1989, 4: 11-17), which has been incorporated into the ALIGN program (version 2.0) using a PAM120 weight residue table, a gap length penalty of 12 and a gap penalty of 4. The percent identity between two nucleotide sequences can, alternatively, be determined for example using the GAP program in the GCG software package using an NWSgapdna.CMP matrix.

Nucleic acid: As used herein, the term “nucleic acid,” in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to individual nucleic acid residues (e.g., nucleotides and/or nucleosides); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester scaffold. For example, in some embodiments, a nucleic acid is, comprises, or consists of one or more “peptide nucleic acids”, which are known in the art and have peptide bonds instead of phosphodiester bonds in the scaffold, are considered within the scope of the present disclosure. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxyguanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid can comprise or consist of one or more inhibitory nucleic acids (e.g., small RNA molecules). In some embodiments, an inhibitory nucleic acid comprises or consists of an RNA molecule (e.g., a small RNA molecule) that inhibits gene expression (e.g., via mRNA degradation) or inhibits translation (e.g., decreases the level of gene expression or translation of a transcript as compared to a relevant control). In some embodiments, an inhibitory nucleic acid comprises or consists of one or more siRNA, miRNA, shRNA, gRNA, or any combination thereof. In some embodiments, an inhibitory nucleic acid can be single stranded or double stranded.

Pharmaceutical composition: As used herein, the term “pharmaceutical composition” refers to an active agent, formulated together with one or more pharmaceutically acceptable carriers. In some embodiments, an active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation: topical application, for example, as a cream, ointment, or a controlled-release patch or spray applied to the skin, lungs, or oral cavity: intravaginally or intrarectally, for example, as a pessary, cream, or foam; sublingually; ocularly; transdermally; or nasally, pulmonary, and to other mucosal surfaces. In some embodiments, a pharmaceutical composition is formulated for subretinal administration, e.g., by subretinal injection.

Recombinant adeno-associated viral (rAAV) particle: A “recombinant adeno-associated viral (rAAV) particle”, or “rAAV particle.” as used herein, refers to an infectious, replication-defective viral particle comprising an AAV protein shell encapsulating at least one payload that is flanked on both sides by inverted terminal repeats (ITRs) in a vector. An rAAV particle can be produced in suitable host cells described herein (e.g., HEK293 cells, CHO-K cells, HeLa cells, or a variant thereof). For example, host cells are transfected with one or more vectors encoding: at least one payload flanked by an ITR on either side of the at least one payload, at least one Rep polypeptide, at least one Cap polypeptide, and at least one helper polypeptide, such that the host cells are capable of producing Rep, Cap and helper polypeptides necessary for packaging of rAAV particles, rAAV particles described herein may be used for subsequent gene delivery.

Subject: As used herein, the term “subject” or “patient” refers to any organism to which a provided composition is or may be administered, e.g., for experimental, diagnostic, prophylactic, cosmetic, and/or therapeutic purposes. In some embodiments, a subject is or comprises a cell or a tissue. Typical patients include animals (e.g., mammals such as mice, rats, rabbits, non-human primates, and/or humans). In some embodiments, a patient is a human. In some embodiments, a patient is suffering from or susceptible to one or more diseases, disorders, or conditions. In some embodiments, a patient displays one or more symptoms of a disease, disorder, or condition. In some embodiments, a patient has been diagnosed with one or more diseases, disorders, or conditions. In some embodiments, the disease, disorder, or condition is retinal degeneration. In some embodiments, the disease, disorder, or condition is retinitis pigmentosa (e.g., retinitis pigmentosa-11 (RP 11)).

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Susceptible to: An individual who is “susceptible to” a disease, disorder and/or condition is one who has a higher risk of developing the disease, disorder and/or condition than does a member of the general public. In some embodiments, an individual who is susceptible to a disease, disorder and/or condition is predisposed to have that disease, disorder and/or condition. In some embodiments, an individual who is susceptible to a disease, disorder and/or condition may not have been diagnosed with the disease, disorder and/or condition. In some embodiments, an individual who is susceptible to a disease, disorder and/or condition may exhibit symptoms of the disease, disorder and/or condition. In some embodiments, an individual who is susceptible to a disease, disorder and/or condition may not exhibit symptoms of the disease, disorder and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

Vector: As used herein, the term “vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In some embodiments, the term “vector” refers to an agent capable of transporting a nucleic acid, wherein the agent comprises the nucleic acid. In some embodiments, a vector comprises or is an agent capable of transporting a nucleic acid.

Wild-type: As used herein, the term “wild-type” has its art-understood meaning that refers to an entity having a structure and/or activity as found in nature in a “normal” (as contrasted with mutant, diseased, altered, etc.) state or context. Those of ordinary skill in the art will appreciate that wild type genes and polypeptides often exist in multiple different forms (e.g., alleles).

### DETAILED DESCRIPTION OF CERTAIN EMBODIMENTS

Among other things, the present disclosure provides various isolated nucleic acids, vectors, rAAVs, and compositions thereof. In some embodiments, isolated nucleic acids, vectors, or rAAVs comprise a nucleic acid sequence encoding a PRPF31 polypeptide. In some embodiments, provided technologies (e.g. isolated nucleic acids, vectors, rAAVs, compositions thereof, or methods thereof) increase expression of PRPF31. Expression of PRPF31 from a nucleic acid sequence encoding PRPF31 may rely on or be increased by other sequence elements, e.g., one or more of inverted terminal repeats (ITRs), a promoter, an enhancer, a woodchuck hepatitis virus post-transcriptional regulatory element (WPRE), a 3′ untranslated region (UTR) element, or a polyadenylation signal. In some embodiments, provided isolated nucleic acids, vectors, or rAAVs comprise various sequence elements, which, among other things, provide improved expression of PRPF31 (e.g., one or both of greater levels and increased stability). In some embodiments, an isolated nucleic acid, vector, or rAAV comprises a 5′ ITR, a promoter, a nucleic acid sequence encoding PRPF31, a WPRE, a polyadenylation signal, and/or a 3′ ITR as described herein.

### PRPF31

In some embodiments, PRPF31 refers to a gene or a gene product thereof (e.g., a nucleic acid (e.g., DNA or RNA), a transcript (e.g., a PRPF31 mRNA), or a protein encoded thereby (e.g., a PRPF31 polypeptide)) from a species, which may be known as PRPF31, PRP31, pre-mRNA-processing factor 31, U4/U6 small nuclear ribonucleoprotein Prp31, or other terms as known to those of skill in the art. Various PRPF31 sequences including variants thereof are readily available to those of skill in the art. Various technologies, e.g., assays, cells, and animal models, have also been reported and can be utilized for characterization or assessment of provided technologies (e.g., one or more of isolated nucleic acids, vectors, rAAV vectors, or methods) in accordance with the present disclosure.

The PRPF31 gene is reported to encode a PRPF31 protein, which primarily localizes to the cellular nucleus in a variety of tissues including those of the eye (e.g., retinal tissue). PRPF31 protein has been reported to act as a splicing factor as a component of the spliceosome as part of the U4/U6.U5 triple small nuclear ribonucleoprotein (tri-snRNP) complex (Makarova, 0. V. et al., EMBO J. 2002 Mar. 1; 21(5):1148-57). The PRPF31 protein reportedly comprises a coiled-coil domain, a Nop domain (which may provide protein and RNA binding capabilities), a flexible loop, and a C-terminal domain (which also comprises a nuclear localization sequence (NLS); Liu, S. et al., Science 2007 Apr. 6; 316(5821):115-20).

Various mutations in PRPF31 have been reported in the literature. See, e.g., Wheway, G. et al., Exp Eye Res. 2020 March; 192:107950. Reported mutations in PRPF31 include a multitude of variants across various intronic and exonic locations in the gene. In some embodiments, a mutation in PRPF31 is in an intron. In some embodiments, a mutation in PRPF31 is in an exon. In some embodiments, a mutation in PRPF31 is at a splice site. In some embodiments, a mutation in PRPF31 is a missense mutation. In some embodiments, a mutation in PRPF31 is a nonsense mutation. In some embodiments, a mutation in PRPF31 is a frameshift mutation. In some embodiments, a mutation in PRPF31 is a loss-of-function mutation. In some embodiments, a mutation in PRPF31 is a large-scale insertion. In some embodiments, a mutation in PRPF31 is a large-scale deletion. In some embodiments, a mutation in PRPF31 is a loss-of-expression mutation.

Studies have shown that mutant PRPF31 may inhibit normal splicing of rhodopsin (RHO) pre-mRNA and thus reduce RHO expression in retinal cells (Yuan, L. et al., J Neurosci. 2005 Jan. 19; 25(3):748-57). Further research indicates that a variety of genes may suffer from incorrect splicing in the presence of mutant PRPF31 and structural abnormalities in cilia formation in retinal cells (Wheway. G. et al., Nat Cell Biol. 2015 August; 17(8):1074-1087; Buskin, A. et al., 2018 Nat Commun. 2018 Oct. 12; 9(1):4234). Current research indicates that PRPF31-associated diseases, disorders, or conditions (e.g., retinitis pigmentosa) may be mechanistically linked to haploinsufficiency from loss-of-function mutations (Abu-Safieh, L. et al., Mol Vis. 2006 Apr. 18; 12:384-8: Rio Frio, T. et al., J Clin Invest. 2008 April; 118(4):1519-31). Accordingly, providing wild-type PRPF31 to subjects through the usage of provided technologies (e.g., one or more of isolated nucleic acids, vectors, rAAVs, compositions thereof, or methods thereof) in the present disclosure may provide treatment of PRPF31-associated diseases, disorders, and conditions.

### PRPF31-Related Diseases, Disorders, and Conditions

Various diseases, disorders, or conditions are reported to be associated with PRPF31 and may be prevented or treated using the provided technologies in the present disclosure. Generally, a disease, disorder, or condition is associated with PRPF31 if the presence, level, activity, or form of PRPF31 or products (e.g., one or both of transcripts and encoded proteins.) thereof correlates with incidence of or susceptibility to the disease, disorder, or condition (e.g., across a relevant population). In some embodiments, a disease, disorder, or condition associated with PRPF31 may be treated or prevented by providing wild-type PRPF31 or products thereof.

Among other things, the present disclosure provides technologies for preventing or treating various diseases, disorders, or conditions. In some embodiments, a disease, disorder, or condition is an inherited disease, disorder, or condition. In some embodiments, a disease, disorder, or condition is autosomal dominant. In some embodiments, a disease, disorder, or condition is a retinopathy. In some embodiments, a disease, disorder, or condition is PRPF31-associated retinopathy. In some embodiments, a disease, disorder, or condition is retinal degeneration. In some embodiments, a disease, disorder, or condition is retinitis pigmentosa (RP). In some embodiments, a disease, disorder, or condition is autosomal dominant retinitis pigmentosa (adRP). In some embodiments, a disease, disorder, or condition is retinitis pigmentosa-11 (RP11).

### Isolated Nucleic Acids

Various isolated nucleic acids can comprise a nucleic acid sequence encoding PRPF31 as provided in the present disclosure. In some embodiments, an isolated nucleic acid is DNA. In some embodiments, an isolated nucleic acid is double-stranded (ds) DNA. In some embodiments, an isolated nucleic acid is single-stranded (ss) DNA. In some embodiments, an isolated nucleic acid is linear. In some embodiments, an isolated nucleic acid is circular. In some embodiments, an isolated nucleic acid is RNA.

In some embodiments, an isolated nucleic acid comprises a nucleic acid sequence encoding a PRPF31 polypeptide. In some embodiments, a PRPF31 polypeptide is a wild-type PRPF31 polypeptide. In some embodiments, a PRPF31 polypeptide is a human PRPF31 polypeptide. In some embodiments, a PRPF31 polypeptide is a wild-type human PRPF31 polypeptide. In some embodiments, a nucleic acid sequence encoding a PRPF31 polypeptide is native human PRPF31 cDNA. In some embodiments, a nucleic acid sequence encoding a PRPF31 polypeptide is codon-optimized human PRPF31 cDNA. In some embodiments, a nucleic acid sequence encoding a PRPF31 polypeptide is or comprises a nucleic acid sequence with about 70% or more sequence identity to SEQ ID NO: 1. In some embodiments, a nucleic acid sequence encoding a PRPF31 polypeptide is or comprises a nucleic acid sequence with about 75% or more sequence identity to SEQ ID NO: 1. In some embodiments, a nucleic acid sequence encoding a PRPF31 polypeptide is or comprises a nucleic acid sequence with about 80% or more sequence identity to SEQ ID NO: 1. In some embodiments, a nucleic acid sequence encoding a PRPF31 polypeptide is or comprises a nucleic acid sequence with about 85% or more sequence identity to SEQ ID NO: 1. In some embodiments, a nucleic acid sequence encoding a PRPF31 polypeptide is or comprises a nucleic acid sequence with about 90% or more sequence identity to SEQ ID NO: 1. In some embodiments, a nucleic acid sequence encoding a PRPF31 polypeptide is or comprises a nucleic acid sequence with about 95% or more sequence identity to SEQ ID NO: 1. In some embodiments, a nucleic acid sequence encoding a PRPF31 polypeptide is or comprises a nucleic acid sequence with about 99% or more sequence identity to SEQ ID NO: 1. In some embodiments, a nucleic acid sequence encoding a PRPF31 polypeptide is or comprises the nucleic acid sequence of SEQ ID NO: 1.

In some embodiments, an isolated nucleic acid comprises a nucleic acid sequence encoding a second polypeptide that is in-frame with a nucleic acid sequence encoding a PRPF31 polypeptide. In some embodiments, the second polypeptide is fused to the PRPF31 polypeptide when the nucleic acid sequence encoding a second polypeptide and the nucleic acid sequence encoding a PRPF31 polypeptide are transcribed. In some embodiments, the second polypeptide is fused to the N-terminus of the PRPF31 polypeptide. In some embodiments, the second polypeptide is fused to the C-terminus of the PRPF31 polypeptide. In some embodiments, the second polypeptide is or comprises one or more epitope tags or portions thereof. Various epitope tags are known in the art, including c-Myc, FLAG, glutathione S-transferase (GST), hemagglutinin (HA), 6×-His, V5, etc. In some embodiments, the second polypeptide is or comprises one or more of c-Myc, FLAG, glutathione S-transferase (GST), hemagglutinin (HA), 6×-His, or V5 epitope tags, or combination thereof. In some embodiments, the second polypeptide is or comprises a V5 epitope tag. In some embodiments, the second polypeptide is or comprises a fluorescent protein or portion thereof. Various fluorescent proteins are known in the art, including cyan fluorescent protein (CFP), green fluorescent protein (GFP), mCherry, red fluorescent protein (RFP), yellow fluorescent protein (YFP), etc.

In some embodiments, an isolated nucleic acid comprises a promoter. In some embodiments, the nucleic acid sequence encoding PRPF31 is operably linked to a promoter that facilitates transcription of the nucleic acid sequence encoding PRPF31. Various promoters are known in the art including the chicken beta-actin (CBA) promoter. CAG promoter, CASI promoter, RPE65 promoter, VMD2 promoter, etc. In some embodiments, a promoter is constitutive or inducible. In some embodiments, a promoter is constitutive. In some embodiments, a promoter is inducible. In some embodiments, a promoter is tissue or cell specific. In some embodiments, a promoter is not tissue or cell specific. In some embodiments, a promoter comprises one or more promoters, enhancers, or other sequence elements. In some embodiments, a promoter comprises a human cytomegalovirus (CMV) enhancer or portion thereof, a chicken beta-actin (CBA) promoter or portion thereof, a human ubiquitin C (UbC) enhancer or portion thereof, a splice donor, a splice acceptor, or a combination thereof. In some embodiments, a promoter comprises a human CMV enhancer or portion thereof. In some embodiments, a promoter comprises a CBA promoter or portion thereof. In some embodiments, a promoter comprises a human UbC enhancer or portion thereof. In some embodiments, a promoter comprises a splice donor. In some embodiments, a promoter comprises a splice acceptor. In some embodiments, a promoter is or comprises a nucleic acid sequence with about 70% or more sequence identity to SEQ ID NO: 4. In some embodiments, a promoter is or comprises a nucleic acid sequence with about 75% or more sequence identity to SEQ ID NO: 4. In some embodiments, a promoter is or comprises a nucleic acid sequence with about 80% or more sequence identity to SEQ ID NO: 4. In some embodiments, a promoter is or comprises a nucleic acid sequence with about 85% or more sequence identity to SEQ ID NO: 4. In some embodiments, a promoter is or comprises a nucleic acid sequence with about 90% or more sequence identity to SEQ ID NO: 4. In some embodiments, a promoter is or comprises a nucleic acid sequence with about 95% or more sequence identity to SEQ ID NO: 4. In some embodiments, a promoter is or comprises a nucleic acid sequence with about 99% or more sequence identity to SEQ ID NO: 4. In some embodiments, a promoter is or comprises the nucleic acid sequence of SEQ ID NO: 4.

In some embodiments, an isolated nucleic acid comprises one or more inverted terminal repeats (ITRs). Various ITRs are known in the art and include adeno-associated virus (AAV) ITRs. AAV ITRs may be derived from AAV1, AAV2, AAV3, AAV4, AAV5, AAV6, AAV7, AAV8, AAV9, AAV10, AAV 11, AAV12, or any combination or variant thereof. In some embodiments, an ITR is a wild-type ITR. In some embodiments, an ITR is a modified or engineered ITR. In some embodiments, an isolated nucleic acid comprises two ITRs. In some embodiments, an isolated nucleic acid comprises a 5′ ITR and a 3′ ITR. In some embodiments, an ITR is an AAV ITR. In some embodiments, a 5′ ITR is an AAV 5′ ITR. In some embodiments, a 3′ ITR is an AAV 3′ ITR. In some embodiments, an ITR is an AAV2 ITR. In some embodiments, a 5′ ITR is an AAV2 5′ ITR. In some embodiments, a 3′ ITR is an AAV2 3′ ITR. In some embodiments, a 5′ ITR is or comprises a nucleic acid sequence with about 70% or more sequence identity to SEQ ID NO: 2. In some embodiments, a 5′ ITR is or comprises a nucleic acid sequence with about 75% or more sequence identity to SEQ ID NO: 2. In some embodiments, a 5′ ITR is or comprises a nucleic acid sequence with about 80% or more sequence identity to SEQ ID NO: 2. In some embodiments, a 5′ ITR is or comprises a nucleic acid sequence with about 85% or more sequence identity to SEQ ID NO: 2. In some embodiments, a 5′ ITR is or comprises a nucleic acid sequence with about 90% or more sequence identity to SEQ ID NO: 2. In some embodiments, a 5′ ITR is or comprises a nucleic acid sequence with about 95% or more sequence identity to SEQ ID NO: 2. In some embodiments, a 5′ ITR is or comprises a nucleic acid sequence with about 99% or more sequence identity to SEQ ID NO: 2. In some embodiments, a 5′ ITR is or comprises the nucleic acid sequence of SEQ ID NO: 2. In some embodiments, a 3′ ITR is or comprises a nucleic acid sequence with about 70% or more sequence identity to SEQ ID NO: 3. In some embodiments, a 3′ ITR is or comprises a nucleic acid sequence with about 75% or more sequence identity to SEQ ID NO: 3. In some embodiments, a 3′ ITR is or comprises a nucleic acid sequence with about 80% or more sequence identity to SEQ ID NO: 3. In some embodiments, a 3′ ITR is or comprises a nucleic acid sequence with about 85% or more sequence identity to SEQ ID NO: 3. In some embodiments, a 3′ ITR is or comprises a nucleic acid sequence with about 90% or more sequence identity to SEQ ID NO: 3. In some embodiments, a 3′ ITR is or comprises a nucleic acid sequence with about 95% or more sequence identity to SEQ ID NO: 3. In some embodiments, a 3′ ITR is or comprises a nucleic acid sequence with about 99% or more sequence identity to SEQ ID NO: 3. In some embodiments, a 3′ ITR is or comprises the nucleic acid sequence of SEQ ID NO: 3.

In some embodiments, an isolated nucleic acid comprises a woodchuck hepatitis virus post-transcriptional regulatory element (WPRE). In some embodiments, a WPRE is or comprises a nucleic acid sequence with about 70% or more sequence identity to SEQ ID NO: 5. In some embodiments, a WPRE is or comprises a nucleic acid sequence with about 75% or more sequence identity to SEQ ID NO: 5. In some embodiments, a WPRE is or comprises a nucleic acid sequence with about 80% or more sequence identity to SEQ ID NO: 5. In some embodiments, a WPRE is or comprises a nucleic acid sequence with about 85% or more sequence identity to SEQ ID NO: 5. In some embodiments, a WPRE is or comprises a nucleic acid sequence with about 90% or more sequence identity to SEQ ID NO: 5. In some embodiments, a WPRE is or comprises a nucleic acid sequence with about 95% or more sequence identity to SEQ ID NO: 5. In some embodiments, a WPRE is or comprises a nucleic acid sequence with about 99% or more sequence identity to SEQ ID NO: 5. In some embodiments, a WPRE is or comprises the nucleic acid sequence of SEQ ID NO: 5.

In some embodiments, an isolated nucleic acid comprises a 3′ untranslated region (UTR) element. Various 3′ UTR elements are known in the art and include AU-rich elements (AREs), CA-rich elements (CAREs), CU-rich elements (CUREs), GU-rich elements (GREs), differentiation control elements (DICEs), miRNA response elements (MREs), polyadenylation signals, etc. In some embodiments, a 3′ UTR element provides particular properties (e.g., one or both of increased expression and increased stability) to an isolated nucleic acid or transcript thereof. In some embodiments, a 3′ UTR element is a polyadenylation signal.

In some embodiments, an isolated nucleic acid comprises a polyadenylation signal. In some embodiments, an isolated nucleic acid sequence comprises a human growth hormone (hGH) polyadenylation signal. In some embodiments, an isolated nucleic acid comprises a beta-globin polyadenylation signal. In some embodiments, an isolated nucleic acid comprises a human beta-globin polyadenylation signal. In some embodiments, a polyadenylation signal is or comprises a nucleic acid sequence with about 70% or more sequence identity to SEQ ID NO: 6. In some embodiments, a polyadenylation signal is or comprises a nucleic acid sequence with about 75% or more sequence identity to SEQ ID NO: 6. In some embodiments, a polyadenylation signal is or comprises a nucleic acid sequence with about 80% or more sequence identity to SEQ ID NO: 6. In some embodiments, a polyadenylation signal is or comprises a nucleic acid sequence with about 85% or more sequence identity to SEQ ID NO: 6. In some embodiments, a polyadenylation signal is or comprises a nucleic acid sequence with about 90% or more sequence identity to SEQ ID NO: 6. In some embodiments, a polyadenylation signal is or comprises a nucleic acid sequence with about 95% or more sequence identity to SEQ ID NO: 6. In some embodiments, a polyadenylation signal is or comprises a nucleic acid sequence with about 99% or more sequence identity to SEQ ID NO: 6. In some embodiments, a polyadenylation signal is or comprises the nucleic acid sequence of SEQ ID NO: 6.

In some embodiments, an isolated nucleic acid comprises a Kozak consensus sequence. Various Kozak consensus sequences are known in the art, see, e.g., Kozak, M. Proc Natl Acad Sci USA. 1990 November; 87(21):8301-5; Kozak, M. J Cell Biol. 1991 November; 115(4):887-903; Kozak, M. Gene. 2002 Oct. 16; 299(1-2):1-34. In some embodiments, a Kozak consensus sequence is located downstream (3′) of a promoter. In some embodiments, a Kozak consensus sequence is located upstream (5′) of a nucleic acid sequence encoding PRPF31. In some embodiments, a Kozak consensus sequence is located downstream (3′) of a promoter and upstream (5′) of a nucleic acid sequence encoding PRPF31.

In some embodiments, an isolated nucleic acid comprises an AAV 5′ ITR, a promoter, a nucleic acid sequence encoding PRPF31, and an AAV 3′ ITR. In some embodiments, an isolated nucleic acid comprises an AAV 5′ ITR, a promoter, a nucleic acid sequence encoding PRPF31, a WPRE, and an AAV 3′ ITR. In some embodiments, an isolated nucleic acid comprises an AAV 5′ ITR, a promoter, a nucleic acid sequence encoding PRPF31, a WPRE, a polyadenylation signal, and an AAV 3′ ITR. In some embodiments, an isolated nucleic acid comprises the nucleic acid sequence of SEQ ID NO: 1, SEQ ID NO: 2, SEQ ID NO:3, and SEQ ID NO: 4. In some embodiments, an isolated nucleic acid comprises the nucleic acid sequence of SEQ ID NO: 1, SEQ ID NO: 2, SEQ ID NO:3, SEQ ID NO: 4, and SEQ ID NO: 5. In some embodiments, an isolated nucleic acid comprises the nucleic acid sequence of SEQ ID NO: 1, SEQ ID NO: 2, SEQ ID NO: 3, SEQ ID NO: 4, SEQ ID NO: 5, and SEQ ID NO: 6. In some embodiments, an isolated nucleic acid is or comprises a nucleic acid sequence with about 70% or more sequence identity to SEQ ID NO: 7. In some embodiments, an isolated nucleic acid is or comprises a nucleic acid sequence with about 75% or more sequence identity to SEQ ID NO: 7. In some embodiments, an isolated nucleic acid is or comprises a nucleic acid sequence with about 80% or more sequence identity to SEQ ID NO: 7. In some embodiments, an isolated nucleic acid is or comprises a nucleic acid sequence with about 85% or more sequence identity to SEQ ID NO: 7. In some embodiments, an isolated nucleic acid is or comprises a nucleic acid sequence with about 90% or more sequence identity to SEQ ID NO: 7. In some embodiments, an isolated nucleic acid is or comprises a nucleic acid sequence with about 95% or more sequence identity to SEQ ID NO: 7. In some embodiments, an isolated nucleic acid is or comprises a nucleic acid sequence with about 99% or more sequence identity to SEQ ID NO: 7. In some embodiments, an isolated nucleic acid is or comprises the nucleic acid sequence of SEQ ID NO: 7.

### Vectors

Various vectors can comprise an isolated nucleic acid as provided in the present disclosure. In some embodiments, a vector is a bacterial artificial chromosome (BAC), a cosmid, a phagemid, a plasmid, or a viral vector. In some embodiments, a vector is a recombinant vector. In some embodiments, a vector is a recombinant BAC, a recombinant cosmid, a recombinant phagemid, a recombinant plasmid, or a recombinant viral vector. Other suitable vectors are known in the art. In some embodiments, a vector is delivered using a suitable carrier, e.g., liposomes, cell-penetrating peptides, antibodies, etc.

### Viral Vectors

In some embodiments, a vector is a viral vector. In some embodiments, a viral vector is a recombinant viral vector. Recombinant viral vectors have become widely used for inserting nucleic acid sequences (e.g., a gene or an inhibitory nucleic acid) into mammalian cells (e.g., human cells). Many forms of viral vectors can be used to deliver a payload (e.g., a payload described herein, e.g., an isolated nucleic acid as described herein) to a cell, tissue, or organism.

Non-limiting examples of recombinant viral vectors include, but are not limited to, adeno-associated virus (AAV), retrovirus (e.g., Moloney murine leukemia virus (MMLV), Harvey murine sarcoma virus, murine mammary tumor virus, or Rous sarcoma virus), adenovirus, SV40-type virus, polyomavirus, Epstein-Barr virus, papilloma virus, herpes virus, vaccinia virus, baculovirus, or polio virus.

In some embodiments, a recombinant viral vector comprises or is a retroviral vector. Retroviruses are enveloped viruses that belong to viral family Retroviridae. Protocols for production of replication-deficient retroviruses are known in the art (See, e.g., Kriegler, M., Gene Transfer and Expression, A Laboratory Manual, W.H. Freeman Co., New York (1990) and Murry, E. J., Methods in Molecular Biology, Vol. 7, Humana Press, Inc., Cliffton, N.J. (1991), each of which is hereby incorporated by reference in its entirety). A number of retroviral systems are known in the art (See, e.g., U.S. Pat. Nos. 5,994,136, 6,165,782, and 6,428,953, each of which is hereby incorporated by reference in its entirety). In some embodiments, a retrovirus comprises or is a lentivirus of Retroviridae family. In some embodiments, a lentivirus comprises or is human immunodeficiency viruses (e.g., HIV-1 or HIV-2), simian immunodeficiency virus (S1V), feline immunodeficiency virus (FIV), equine infections anemia (EIA), or visna virus.

In some embodiments, a recombinant viral vector comprises or is an adenovirus vector. An adenovirus vector may be from any origin, subgroup, subtype, serotype, or mixture thereof. For instance, an adenovirus can be of subgroup A (e.g., serotypes 12, 18, or 31), subgroup B (e.g., serotypes 3, 7, 11, 14, 16, 21, 34, 35, or 50), subgroup C (e.g., serotypes 1, 2, 5, or 6), subgroup D (e.g., serotypes 8, 9, 10, 13, 15, 17, 19, 20, 22-30, 32, 33, 36-39, or 42-48), subgroup E (e.g., serotype 4), subgroup F (e.g., serotypes 40 or 41), an unclassified serogroup (e.g., serotypes 49 or 51), or any other adenoviral serotype. Adenoviral serotypes I through 51 are available from the American Type Culture Collection (ATCC, Manassas, VA, USA).

Non-group C adenoviruses, and even non-human adenoviruses, can be used to prepare replication-deficient adenoviral vectors. Non-group C adenoviral vectors, methods of producing non-group C adenoviral vectors, and methods of using non-group C adenoviral vectors are disclosed in, for example, U.S. Pat. Nos. 5,801,030, 5,837,511, and 5,849,561, and International Patent Applications WO 97/12986 and WO 98/53087, each of which is hereby incorporated by reference in its entirety. Further examples of adenoviral vectors can be found in U.S. Publication Nos. 20150093831, 20140248305, 20120283318, 20100008889, 20090175897 and 20090088398, each of which is hereby incorporated by reference in its entirety.

In some embodiments, a recombinant viral vector comprises or is an alphavirus. Exemplary alphaviruses include, but are not limited to, Sindbis virus, Aura virus, Babanki virus, Barmah Forest virus, Bebaru virus, Cabassou virus, Chikungunya virus, Eastern equine encephalitis virus, Everglades virus, Fort Morgan virus, Getah virus, Highlands J virus, Kyzylagach virus, Mayaro virus, Me Tri virus, Middelburg virus, Mosso das Pedras virus, Mucambo virus, Ndumu virus, O'nyong-nyong virus, Pixuna virus, Rio Negro virus, Ross River virus, Salmon pancreas disease virus, Semliki Forest virus, Southern elephant seal virus, Tonate virus, Trocara virus, Una virus, Venezuelan equine encephalitis virus, Western equine encephalitis virus, and Whataroa virus. Generally, a genome of such viruses encodes nonstructural (e.g., replicon) and structural proteins (e.g., capsid and envelope) that can be translated in host cell cytoplasm. Ross River virus, Sindbis virus, Semliki Forest virus (SFV), and Venezuelan equine encephalitis virus (VEEV) have all been used to develop viral transfer vectors for transgene delivery. Pseudotyped viruses may be formed by combining alphaviral envelope glycoproteins and retroviral capsids. Examples of alphaviral vectors can be found in U.S. Publication Nos. 20150050243, 20090305344, and 20060177819, each of which is incorporated herein by reference in their entirety

In some embodiments, a recombinant viral vector comprises or is an AAV vector. AAV systems are generally well known in the art (see, e.g., Kelleher and Vos, Biotechniques, 17(6):1110-17 (1994); Cotten et al., P.N.A.S. U.S.A., 89(13):6094-98 (1992); Curiel. Nat Immun, 13(2-3):141-64 (1994); Muzyczka, Curr Top Microbiol Immunol, 158:97-129 (1992); and Asokan A. et al., Mol. Ther., 20(4):699-708 (2012), each of which is hereby incorporated by reference in its entirety). Methods for generating and using AAV vectors are described, for example, in U.S. Pat. Nos. 5,139,941 and 4,797,368, each of which is hereby incorporated by reference in its entirety.

Generally, AAV vectors for use in methods, compositions, and systems described herein may be of any AAV serotype. AAV serotypes generally have different tropisms to infect different tissues. In some embodiments, an AAV serotype is selected based on a tropism. Several AAV serotypes have been characterized including, but not limited to, AAV1, AAV2, AAV3A, AAV3B, AAV4, AAV5, AAV6, AAV7, AAV8, AAV9, AAV10, AAV11, AAVrh10, AAVrh74, AAV-HSC 1-17, AAV-CBr, AAV-CLv, AAV-CLg, AAV-DJ, AAV-PHP.B, AAV-PHP.N, or AAV.CAP-B1 to AAV.CAP-B25, as well as variants or hybrids thereof.

In some embodiments, an AAV vector is derived from an AAV genome sequence or a variant thereof as described in U.S. Pat. Nos. 7,906,111; 6,759,237; 7,105,345; 7,186,552; 9,163,260; 9,567,607; 4,797,368; 5,139,941; 5,252,479; 6,261,834; 7,718,424; 8,507,267; 8,846,389; 6,984,517; 7,479,554; 6,156,303; 8,906,675; 7,198,951; 10,041,090; 9,790,472; 10,308,958; 10,526,617; 7,282,199; 7,790,449; 8,962,332; 9,587,250; 10,590,435; 10,265,417; 10,485,883; 7,588,772; 8,067,01; 8,574,583; 8,906,387; 8,734,809; 9,284,357; 10,035,825; 8,628,966; 8,927,514; 9,623,120; 9,777,291; 9,783.825; 9,803,218; 9,834,789; 9,839.696; 9,585,971; or 10,519,198; U.S. Publication Nos. 2017/0166926; 2019/0015527; 2019/0054188; or 2020/0080109; or International Publication Nos. WO2018/160582, WO2020/028751, or WO2020/068990, each of which is hereby incorporated by reference in its entirety.

In some embodiments, an AAV vector comprises or is a single-stranded (ss) or self-complementary (sc) AAV vector. In some embodiments, an AAV vector comprises an expression construct and one or more regions comprising ITR sequences (e.g., wild-type ITR sequences or engineered or modified ITR sequences) flanking an expression construct. In some embodiments, an expression construct comprises an enhancer, a promoter, a nucleic acid sequence encoding a product of interest (e.g., a polypeptide), a WPRE, or a 3′ UTR element (e.g., a polyadenylation signal), or a combination thereof.

### Adeno-Associated Viruses (AAVs)

Various adeno-associated viruses (AAVs) are provided herein. In some embodiments, the present disclosure provides recombinant AAVs (rAAVs). In some embodiments, the present disclosure provides rAAVs comprising (i) a capsid and (ii) an isolated nucleic acid as described herein or a vector as described herein.

AAV is reportedly a small, non-enveloped virus that packages a single-stranded, linear DNA genome, approximately 4.7-5 kb long. A member of the family Parvoviridae, AAV was discovered in 1965 as a contaminant of adenovirus isolates. AAV has not been associated with any human or animal disease, even though most humans (>70%) are seropositive for one or more serotypes (Calcedo et al. (2011); Calcedo et al. (2009)). Both positive and negative DNA strands are packaged equally well, and infection can be initiated with particles containing either strand. The virus has a T=1 icosahedral capsid, 25 nm in diameter, that is reportedly extraordinarily stable. It has been demonstrated to resist brief exposure to heat, acidic pH, and proteases. The viral genome comprises three open reading frames (ORFs), rep (replication), cap (capsid), and aap (assembly-activating protein), which together code for eight proteins (Rep78, Rep68, Rep52, Rep40, VP1, VP2, VP3, and AAP) expressed from three promoters (p5, p19, and p40). The mature capsid consists of the amino acid sequence of only one ORF (cap) and the packaged DNA. Thus, recombinant AAVs (rAAVs) may present as a small target for the host immune system.

### Inverted Terminal Repeats (ITRs)

The present disclosure recognizes that the coding regions of AAV are flanked by inverted terminal repeats (ITRs) that are typically 145 bases long in wild-type AAVs and have a complex T-shaped structure. These repeats are the origins for DNA replication and serve as the primary packaging signal (McLaughlin et al., 1988; Hauswirth et al., 1977). The present disclosure further recognizes that ITRs are the only cis-active sequences required for making rAAVs and the only AAV-encoded sequences present in AAV vectors (McLaughlin et al. (1988); Samulski et al. (1989)). Although AAV ITRs have enhancer activity in the presence of Rep protein, they have minimal promoter or enhancer activity in the absence of Rep protein. Thus, transgenes cloned into an AAV vector must be engineered with an appropriate enhancer, promoter, polyadenylation signal, and/or splice sites to ensure correct gene expression.

Various ITRs are provided by the present disclosure. In some embodiments, ITRs of the present disclosure can include ITRs from any AAV serotype. In some embodiments. ITRs of the present disclosure can include ITRs from AAV1, AAV2, AAV3, AAV4, AAV5, AAV6, AAV7, AAV8, AAV9, AAV10, AAV 11, AAV12, or any combination thereof. In some embodiments, ITRs of the present disclosure may comprise engineered or modified ITRs using methods known in the art. In some embodiments, ITRs of the present disclosure may comprise one or more sequence modifications (e.g., deletions, substitutions) as compared to a wild-type ITR sequence.

ITRs of an AAV vector can be derived from any AAV serotype (e.g., AAV1, AAV2, AAV3A, AAV3B, AAV4, AAV5, AAV6, AAV7, AAV8, AAV9, AAV10, AAV11, AAVrh10, AAVrh74, AAV-HSC 1-17, AAV-CBr. AAV-CLv. AAV-CLg. AAV-DJ, AAV-PHP.B, AAV-PHP.N, or AAV.CAP-B1 to AAV.CAP-B25, or variants or hybrids thereof). In some embodiments, ITRs are derived from one or more other serotypes, e.g., as described in U.S. Pat. Nos. 7,906,111; 6,759,237; 7,105,345; 7,186,552; 9,163,260; 9,567,607; 4,797,368; 5,139,941; 5,252,479; 6,261,834; 7,718,424; 8,507,267; 8,846,389; 6,984,517; 7,479,554; 6,156,303; 8,906,675; 7,198,951; 10,041,090; 9,790,472; 10,308,958; 10,526,617; 7,282,199; 7,790,449; 8,962,332; 9,587,250; 10,590,435; 10,265,417; 10,485,883; 7,588,772; 8,067,01; 8,574,583; 8,906,387; 8,734,809; 9,284,357; 10,035,825; 8,628,966; 8,927,514; 9,623,120; 9,777,291; 9,783,825; 9,803,218; 9,834,789; 9,839,696; 9,585,971; or 10,519,198; U.S. Publication Nos. 2017/0166926; 2019/0015527; 2019/0054188; or 2020/0080109; or International Publication Nos. WO2018/160582, WO2020/028751, or WO2020/068990, each of which is hereby incorporated by reference in its entirety.

ITR sequences and plasmids containing ITR sequences are known in the art and are commercially available (See, e.g., products and services available from Vector Biolabs. Philadelphia. PA; Cellbiolabs, San Diego, CA; Agilent Technologies, Santa Clara, Ca; and Addgene, Cambridge, MA; and described in Kessler et al., PNAS. 1996 Nov. 26; 93(24): 14082-7: Machida. Methods in Molecular Medicine™. Viral Vectors for Gene Therapy Methods and Protocols. 10.1385/1-59259-304-6:201 © Humana Press Inc. 2003. Chapter 10. Targeted Integration by Adeno-Associated Virus; and U.S. Pat. Nos. 5,139,941 and 5,962,313; each of which is hereby incorporated by reference in its entirety).

### Capsids

The present disclosure encompasses the recognition that more than 110 distinct primate AAV capsid sequences have been reportedly isolated. Each of those AAV capsids that have unique serological profiles has been named as a particular AAV serotype. The present disclosure further appreciates that at least 12 primate serotypes (e.g., AAV1, AAV2, AAV3, AAV4, AAV5, AAV6, AAV7, AAV8, AAV9, AAV10, AAV 11, AAV12) have been described. In some embodiments of the present disclosure, a capsid from any serotype can be used. In some embodiments, a modified or engineered capsid including, but not limited to those described herein, can be used in accordance with the present disclosure.

The present disclosure recognizes that numerous studies have evaluated and compared serotypes with regard to their transduction efficiency in tissues in vivo. For example, in striated muscle, studies achieved high transduction efficiency with AAV1, AAV6, and AAV7. Similarly, AAV8 and AAV9 have been found to transduce striated muscle with efficiencies at least as high. AAV8 and AAV9 are considered to have the highest level of hepatocyte transduction. In the pulmonary system, rAAV6 and rAAV9 transduce much of the entire airway epithelium, while rAAV5 transduction is limited to lung alveolar cells. With respect to transduction of the central nervous system, rAAV serotypes 1, 4, 5, 7, and 8 have been found to be efficient transducers of neurons in various regions of the brain, rAAV1 and rAAV5 have also been reported to transduce ependymal and glial cells. In the eye, rAAV serotypes 1, 4, 5, 7, 8, and 9 efficiently transduce retinal pigmented epithelium, while rAAV5, rAAV7, and rAAV8 transduce photoreceptors as well. rAAV1, rAAV8, and rAAV9 have shown the highest reported transduction in pancreas tissue, primarily in acinar cells. The kidney appears to be a relatively difficult organ to transduce, although proximal tubule cells have been transduced by rAAV2 at low levels, as have glomeruli by rAAV9. Additionally, rAAV1 has been shown to transduce adipose tissue, albeit with the aid of a nonionic surfactant.

The present disclosure additionally encompasses the recognition that it may be advantageous to modify wild type AAV capsids, or engineer AAV capsids, to achieve modified tissue tropism and/or immune system evasion. One method of achieving these advantages is to produce vector in the presence of cap genes for multiple serotypes. Depending on the ratio of capsid proteins from each serotype, the resulting “mosaic” virions can exhibit a combined tropism for cell type or, in some cases, can acquire tropism not exhibited by either serotype individually. Some studies have involved attaching exogenous molecules to the capsid. One example utilizes a bi-specific antibody obtained by fusing Fc regions of two different antibodies: an anti-capsid antibody and an anti-cell marker antibody, thereby conferring rAAV2 tropism to transduction-resistant megakaryocyte cell lines. Another example adopted the approach of biotinylating the capsid and subsequently binding it to a streptavidin conjugate carrying epidermal growth factor or fibroblast growth factor. This approach was shown to produce at least a ten-fold increase in the transduction of cells that highly express the epidermal growth factor or fibroblast growth factor receptor, respectively.

The present disclosure also appreciates that as an alternative to attaching molecules to the capsid surface, it may be advantageous to engineer a modification directly into the cap gene. As one non-limiting example, green fluorescent protein (GFP) (238 amino acids) can be inserted into AAV2 VP1 and VP2. Although the transduction efficiencies of the VP1-GFP and VP2-GFP vectors were 3 and 5 orders of magnitude lower, respectively, than the efficiency of wild-type capsid, the transduction in HeLa cells did occur, suggesting a tolerance for inserted sequences in capsid proteins. As another non-limiting example, for modifying cap genes for tissue targeting, a number of researchers have inserted peptide sequences on the basis of known ligand-receptor interactions, or have selected for peptides in phage-display libraries. Another strategy has been to insert random sequences of amino acids, followed by in vitro selection of the best performing capsids. Instead of introducing target-specific peptides, some experiments modified the capsids generically, pending subsequent modification toward targets of choice. For example, a binding site for the Fe portion of antibodies was inserted into the capsid, followed by binding of different antibodies specific for receptors of various cell lines. Another such modification is to insert a biotin-binding site into the capsid, thereby facilitating metabolic biotinylation and allowing flexible targeting with any avidin-conjugated ligands. Some experiments have taken advantage of peptide insertion as well as mosaic capsids with a virion containing both wild-type capsid proteins and engineered capsid proteins, or a virion containing a combination of multiple different modified capsid proteins. Other techniques are under investigation with a view to evading the immune system, and these include coating capsids with polymer.

### Production

Methods of producing and isolating rAAV with a desired isolated nucleic acid sequence or vector, and capsid are well known in the art. rAAV of the present disclosure can be produced and isolated according to any appropriate method, e.g., methods described in Clement and Grieger, 2016, Grieger et al., 2016, and Martin et al., 2013, the contents of each which are incorporated herein by reference in their entirety. Without wishing to be bound by any particular theory or process, the methods typically involve culturing a host cell which contains a nucleic acid sequence (e.g., a cap gene) encoding an AAV capsid protein or fragment thereof; a functional rep gene; a nucleic acid sequence or vector comprising AAV ITRs (e.g., an AAV 5′ ITR and an AAV 3′ ITR) and a nucleic acid sequence encoding a product of interest (e.g., a polypeptide. e.g., a wild-type polypeptide); and sufficient helper functions to permit packaging of the recombinant AAV vector into the AAV capsid proteins.

The components to be cultured in the host cell to package an isolated nucleic acid sequence or vector in an AAV capsid may be provided to the host cell in trans. Alternatively, any one or more of the required components (e.g., isolated nucleic acid sequence or vector, rep sequences, cap sequences, and/or helper functions) may be provided by a stable host cell which has been engineered to contain one or more of the required components using methods known to those of skill in the art. Most suitably, such a stable host cell will contain the required component or components under the control of an inducible promoter. However, the required component or components may be under the control of a constitutive promoter. Examples of suitable promoters are provided herein. In still another alternative, a selected stable host cell may contain a selected component or components under the control of a constitutive promoter and other selected component or components under the control of one or more inducible promoters. For example, a stable host cell may be generated which is derived from 293 cells (which contain E1 helper functions under the control of a constitutive promoter), but which contain the rep and/or cap proteins under the control of inducible promoters. Still other stable host cells are known in the art or may be generated by one of skill in the art.

The isolated nucleic acid sequence or vector, rep sequences, cap sequences, and helper functions required for producing the rAAV of the disclosure may be delivered to the packaging host cell using any appropriate genetic element (e.g., a vector). The selected genetic element may be delivered by any suitable method (e.g., transfection), including those described herein. The methods used to construct any embodiment of this disclosure are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al., 1993 and U.S. Pat. No. 5,478,745.

In some embodiments, rAAVs may be produced using the triple transfection method (e.g., as described in detail in U.S. Pat. No. 6,001,650, the contents of which relating to the triple transfection method are incorporated herein by reference). Typically, the rAAVs are produced by transfecting a host cell with a suitable vector (comprising a nucleic acid sequence encoding a product of interest, e.g., a polypeptide) to be packaged into rAAV particles, an AAV rep/cap vector, and a helper function vector. An AAV rep/cap vector encodes rep and cap sequences, which function in trans for productive AAV replication and encapsidation. In some embodiments, the AAV rep/cap vector supports efficient AAV vector production without generating any detectable wild-type AAV virions (e.g., AAV virions containing functional rep and cap genes). Non-limiting examples of vectors suitable for use with the present disclosure include pHLP19, described in U.S. Pat. No. 6,001,650 and pRep6cap6 vector, described in U.S. Pat. No. 6,156,303, the entirety of both incorporated by reference herein. The helper function vector encodes nucleotide sequences for non-AAV derived viral and/or cellular functions upon which AAV is dependent for replication (e.g., “helper functions”). The helper functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based helper functions can be derived from any of the known helper viruses such as adenovirus, herpesvirus (other than herpes simplex virus type-1), and vaccinia virus.

### Recombinant Viral Particles

The present disclosure, among other things, provides methods, compositions, and systems comprising or for producing recombinant viral particles (e.g., recombinant adeno-associated viral (rAAV) particles). In some embodiments, a rAAV particle comprises an isolated nucleic acid as described herein. In some embodiments, a rAAV particle comprises a vector as described herein. In some embodiments, a rAAV particle comprises an AAV genome and a capsid. In some embodiments, a rAAV particle comprises an AAV genome comprising an isolated nucleic acid as described herein and a capsid. In some embodiments, a rAAV particle comprises an AAV genome comprising a vector as described herein and a capsid. In some embodiments, a rAAV particle comprises a modified AAV genome comprising a nucleic acid sequence encoding a polypeptide and a capsid. In some embodiments, a rAAV particle comprises a modified AAV genome comprising (i) a promoter. (ii) a nucleic acid sequence encoding a polypeptide, and (iii) a WPRE; and a capsid. In some embodiments, a rAAV particle comprises a modified AAV genome comprising (i) a 5′ ITR, (ii) a promoter, (iii) a nucleic acid sequence encoding a polypeptide, (iv) a WPRE, and (v) a 3′ ITR; and a capsid. In some embodiments, a rAAV particle comprises a modified AAV genome comprising (i) a 5′ ITR, (ii) a promoter, (iii) a nucleic acid sequence encoding a polypeptide, (iv) a WPRE, (v) a 3′ UTR element, and (vi) a 3′ ITR; and a capsid. In some embodiments, a polypeptide is PRPF31. In some embodiments, a polypeptide is wild-type PRPF31.

In some embodiments, an AAV serotype may have or comprise a mutation in an AAV2 sequence (e.g., as described in Wu et al., J Virol. 2000 September; 74(18):8635-47, which is hereby incorporated by reference in its entirety). Other AAVs are described in, e.g., Sharma et al., Brain Res Bull. 2010 Feb. 15; 81(2-3): 273, which is hereby incorporated by reference in its entirety.

In some embodiments, an AAV comprises or is a naturally occurring AAV. In some embodiments, an AAV is a modified AAV or a variant of a naturally occurring AAV. In some embodiments, an AAV may be generated by directed evolution, e.g., by DNA shuffling, peptide insertion, or random mutagenesis, in order to introduce modifications into the AAV sequence to improve one or more properties for gene therapy. In some embodiments, such modifications avoid or lessen an immune response or recognition by neutralizing antibodies and/or allow for more efficient and/or targeted transduction (See, e.g., Asuri et al., Molecular Therapy 20.2 (2012): 329-338, which is hereby incorporated by reference in its entirety). Methods of using directed evolution to engineer an AAV can be found. e.g., in U.S. Pat. No. 8,632,764, which is hereby incorporated by reference in its entirety. In some embodiments, a modified AAV is modified to include a specific tropism.

In some embodiments, an AAV may be a dual or triple AAV composition, e.g., for the delivery of large payloads (e.g., payloads of greater than approximately 5kb) and/or to address safety concerns associated with administration of single AAV particles. In some embodiments, a dual AAV composition may include two separate AAV particles, each including a fragment of a full sequence of a large payload of interest, and when recombined, the fragments form the full sequence of the large payload of interest or a functional portion thereof. In some embodiments, a triple AAV composition may include three separate AAV particles, each including a fragment of a sequence of a large payload of interest, and when recombined, the fragments form the full sequence of the large payload of interest or a functional portion thereof.

Multiple AAV (e.g., dual or triple AAV compositions) can be delivered to and co-transduced into the same cell, where fragments of a payload of interest recombine and generate a single mRNA transcript of the entire payload of interest. In some embodiments, fragmented payloads include non-overlapping sequences. In some embodiments, fragmented payloads include one or more specified overlapping sequences. In some embodiments, multiple AAVs for dual or triple transfection may be the same type of AAV (e.g., same serotype and/or same construct). In some embodiments, multiple AAVs for dual or triple transfection may be different types of AAV (e.g., different serotype and/or different construct).

In some embodiments, a rAAV comprises an isolated nucleic acid or a vector (e.g., an AAV vector) as described herein. In some embodiments, a rAAV comprises an AAV vector encapsidated by a viral capsid. In some embodiments, a viral capsid comprises 60 capsid protein subunits. In some embodiments, a viral capsid comprises VP1, VP2, and VP3. In some embodiments, VP1, VP2, and VP3 subunits are present in a capsid at a ratio of about 1:1:10, respectively.

A rAAV may comprise or be based on a serotype selected from any following serotypes or variants thereof including, but not limited to, AAV9.68, AAV1, AAV10, AAV106.1/hu.37, AAV11, AAV114.3/hu.40, AAV 12, AAV127.2/hu.41, AAV127.5/hu.42, AAV128.1/hu.43, AAV128.3/hu.44, AAV130.4/hu.48, AAV145.1/hu.53, AAV145.5/hu.54, AAV145.6/hu.55, AAV16.12/hu.11, AAV16.3, AAV16.8/hu.10, AAV161.10/hu.60, AAV161.6/hu.61, AAV1-7/rh.48, AAV1-8/rh.49, AAV2, AAV2.5T, AAV2-15/rh.62, AAV223.1, AAV223.2, AAV223.4, AAV223.5, AAV223.6, AAV223.7, AAV2-3/rh.61, AAV24.1, AAV2-4/rh.50, AAV2-5/rh.51, AAV27.3, AAV29.3/bb. 1, AAV29.5/bb.2, AAV2G9, AAV-2-pre-miRNA-101, AAV3, AAV3.1/hu.6, AAV3.1/hu.9, AAV3-11/rh.53, AAV3-3, AAV33.12/hu.17, AAV33.4/hu.15, AAV33.8/hu.16, AAV3-9/rh.52, AAV3a, AAV3b, AAV4, AAV4-19/rh.55, AAV42.12, AAV42-10, AAV42-11, AAV42-12, AAV42-13, AAV42-15, AAV42-1b, AAV42-2, AAV42-3a, AAV42-3b, AAV42-4, AAV42-5a, AAV42-5b, AAV42-6b, AAV42-8, AAV42-aa, AAV43-1, AAV43-12, AAV43-20, AAV43-21, AAV43-23, AAV43-25, AAV43-5, AAV4-4, AAV44.1, AAV44.2, AAV44.5, AAV46.2/hu.28, AAV46.6/hu.29, AAV4-8/r11.64, AAV4-8/rh.64, AAV4-9/rh.54, AAV5, AAV52.1/hu.20, AAV52/hu.19, AAV5-22/rh.58, AAV5-3/rh.57, AAV54.1/hu.21, AAV54.2/hu.22, AAV54.4R/hu.27, AAV54.5/hu.23, AAV54.7/hu.24, AAV58.2/hu.25, AAV6, AAV6.1, AAV6.1.2, AAV6.2, AAV7, AAV7.2, AAV7.3/hu.7, AAV8, AAV-8b, AAV-8h, AAV9, AAV9.11, AAV9.13, AAV9.16, AAV9.24, AAV9.45, AAV9.47, AAV9.61, AAV9.84, AAV9.9, AAVA3.3, AAVA3.4, AAVA3.5, AAV A3.7, AAV-b, AAVC1, AAVC2, AAVC5, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAV-DJ, AAV-DJ8, AAVF3, AAVF5, AAV-h, AAVH-1/hu.1, AAVH2, AAVH-5/hu.3, AAVH6, AAVhE1.1, AAVhER1.14, AAVhEr1.16, AAVhEr1.18, AAVhER1.23, AAVhEr1.35, AAVhEr1.36, AAVhEr1.5, AAVhEr1.7, AAVhEr1.8, AAVhEr2.16, AAVhEr2.29, AAVhEr2.30, AAVhEr2.31, AAVhEr2.36, AAVhEr2.4, AAVhEr3.1, AAVhu.1, AAVhu.10, AAVhu.11, AAVhu.12, AAVhu.13, AAVhu.14/9, AAVhu.15, AAVhu.16, AAVhu.17, AAVhu.18, AAVhu.19, AAVhu.2, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.3, AAVhu.31, AAVhu.32, AAVhu.34, AAVhu.35, AAVhu.37, AAVhu.39, AAVhu.4, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.5, AAVhu.51, AAVhu.52, AAVhu.53, AAVhu.54, AAVhu.55, AAVhu.56, AAVhu.57, AAVhu.58, AAVhu.6, AAVhu.60, AAVhu.61, AAVhu.63, AAVhu.64, AAVhu.66, AAVhu.67, AAVhu.7, AAVhu.8, AAVhu.9, AAVhu.t19, AAVLG-10/rh.40, AAVLG-4/rh.38, AAVLG-9/hu.39, AAVLG-9/hu.39, AAV-LK01, AAV-LK02, AAVLK03, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK17, AAV-LK18, AAV-LK19, AAVN721-8/rh.43, AAV-PAEC, AAV-PAEC11, AAV-PAEC12, AAV-PAEC2, AAV-PAEC4, AAV-PAEC6, AAV-PAEC7, AAV-PAEC 8, AAVpi.1, AAVpi.2, AAVpi.3, AAVrh.10, AAVrh.12, AAVrh.13, AAVrh.13R, AAVrh.14, AAVrh.17, AAVrh.18, AAVrh.19, AAVrh.2, AAVrh.20, AAVrh.21, AAVrh.22, AAVrh.23, AAVrh.24, AAVrh.25, AAVrh.2R, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.43, AAVrh.44, AAVrh.45, AAVrh.46, AAVrh.47, AAVrh.48, AAVrh.48, AAVrh.48.1, AAVrh.48.1.2, AAVrh.48.2, AAVrh.49, AAVrh.50, AAVrh.51, AAVrh.52, AAVrh.53, AAVrh.54, AAVrh.55, AAVrh.56, AAVrh.57, AAVrh.58, AAVrh.59, AAVrh.60, AAVrh.61, AAVrh.62, AAVrh.64, AAVrh.64R1, AAVrh.64R2, AAVrh.65, AAVrh.67, AAVrh.68, AAVrh.69, AAVrh.70, AAVrh.72, AAVrh.73, AAVrh.74, AAVrh.8, AAVrh.8R, AAVrh8R, AAVrh8R A586R mutant, AAVrh8R R533A mutant, BAAV, B P61 AAV, B P62 AAV, B P63 AAV, bovine AAV, caprine AAV, Japanese AAV10, true type AAV (ttAAV), UPENN AAV 10, AAV-LK 16, AAAV, AAV Shuffle 100-1, AAV Shuffle 100-2, AAV Shuffle 100-3, AAV Shuffle 100-7, AAV Shuffle 10-2, AAV Shuffle 10-6, AAV Shuffle 10-8, AAV SM 100-10, AAV SM 100-3, AAV SM 10-1, AAV SM 10-2, and AAV SM 10-8.

An AAV serotype may be from any number of species. For example, an AAV may be or comprise an avian AAV (AAAV), e.g., as described in U.S. Pat. No. 9,238,800, which is hereby incorporated by reference in its entirety. An AAV serotype may be or comprise a bovine AAV (BAAV), e.g., as described in U.S. Pat. Nos. 9,193,769 or 7,427,396, each of which is hereby incorporated by reference in its entirety. An AAV may be or comprise a caprine AAV, e.g., as described in U.S. Pat. No. 7,427,396, which is hereby incorporated by reference in its entirety. An AAV serotype may also be a variant or hybrid of any of the foregoing. In some embodiments, a rAAV may be or comprise a serotype generated from an AAV2 capsid library.

In some embodiments, a rAAV comprises a capsid including modified capsid proteins (e.g., capsid proteins comprising a modified VP3 region). Methods of producing modified capsid proteins are known in the art (See, e.g., US20130310443, which is hereby incorporated by reference in its entirety). In some embodiments, a rAAV comprises a modified capsid protein comprising at least one non-native amino acid substitution at a position that corresponds to a surface-exposed amino acid (e.g., a surface exposed tyrosine) in a wild-type capsid protein. In some embodiments, a rAAV comprises a modified capsid protein comprising a non-tyrosine amino acid (e.g., a phenylalanine) at a position that corresponds to a surface-exposed tyrosine amino acid in a wild-type capsid protein, a non-threonine amino acid (e.g., a valine) at a position that corresponds to a surface-exposed threonine amino acid in a wild-type capsid protein, a non-lysine amino acid (e.g., a glutamic acid) at a position that corresponds to a surface-exposed lysine amino acid in a wild-type capsid protein, a non-serine amino acid (e.g., a valine) at a position that corresponds to a surface-exposed serine amino acid in a wild-type capsid protein, or a combination thereof. In some embodiments, a rAAV comprises a capsid that includes modified capsid proteins having at least 1, 2, 3, 4, 5, 6, 7, 8, 9, 10, or more amino acid substitutions.

Additional methods for generating and isolating rAAVs suitable for delivery to a subject are described in, e.g., U.S. Pat. Nos. 7,790,449; 7,282,199; WO 2003/042397; WO 2005/033321, WO 2006/110689; and U.S. Pat. No. 7,588,772, each of which are hereby incorporated by reference in their entirety.

### Characterization and Assessment

In some embodiments, properties and/or activities of provided isolated nucleic acids, vectors, rAAVs, and compositions thereof can be characterized and/or assessed using various technologies available to those skilled in the art, e.g., biochemical assays, cell-based assays, animal models, or clinical trials. Certain useful technologies are described in the Examples. Those skilled in the art reading the present disclosure will readily appreciate that other technologies, e.g., in vitro models (e.g., cell lines) for various diseases, disorders, or conditions, animal models for various diseases, disorders, or conditions, etc. may be designed and/or utilized to assess provided technologies (e.g., isolated nucleic acids, vectors, rAAVs, compositions, or methods.) in accordance with the present disclosure.

### Biological Applications

As appreciated by those skilled in the art, isolated nucleic acids, vectors, and rAAVs are useful for many purposes. In some embodiments, provided technologies (e.g., isolated nucleic acids, vectors, rAAVs, compositions thereof, methods thereof) are useful for increasing levels and/or activities of PRPF31 transcripts (e.g., mRNA) and/or products encoded thereby (e.g., polypeptides and/or proteins). In some embodiments, provided technologies increase levels and/or activities of wild-type PRPF31 transcripts. In some embodiments, provided technologies increase levels and/or activities of wild-type PRPF31 polypeptides.

In some embodiments, the present disclosure provides a method of increasing the expression of PRPF31 in a system, comprising administering or delivering to the system an isolated nucleic acid, vector, rAAV, or composition thereof as provided herein. In some embodiments, the present disclosure provides a method of increasing expression of PRPF31 in a system, comprising administering or delivering to the system an effective amount of an isolated nucleic acid, vector, rAAV, or composition thereof as provided herein. In some embodiments, the present disclosure provides a method of increasing levels of PRPF31 polypeptide in a system, comprising administering or delivering to the system an isolated nucleic acid, vector, rAAV, or composition thereof as provided herein. In some embodiments, the present disclosure provides a method of increasing levels of PRPF31 polypeptide in a system, comprising administering or delivering to the system an effective amount of an isolated nucleic acid, vector, rAAV, or composition thereof as provided herein. In some embodiments, the present disclosure provides a method of increasing phagocytosis by cells of the retinal pigment epithelium (RPE), comprising administering or delivering to the system an isolated nucleic acid, vector, rAAV, or composition thereof as provided herein. In some embodiments, the present disclosure provides a method of increasing phagocytosis by cells of the retinal pigment epithelium (RPE), comprising administering or delivering to the system an effective amount of an isolated nucleic acid, vector, rAAV, or composition thereof as provided herein. In some embodiments, phagocytosis is quantified by the number of phagosomes in the retinal pigment epithelium (RPE). In some embodiments, phagocytosis is quantified by the binding and internalization of a payload, e.g., a fluorescent marker.

In some embodiments, a system is an in vitro system. In some embodiments, a system is an in vivo system. In some embodiments, a system comprises a cell. In some embodiments, a system is a cell. In some embodiments, a system comprises a population of cells. In some embodiments, a system is a population of cells. In some embodiments, a cell is a cell in the eye. In some embodiments, a cell is a retinal cell. In some embodiments, a cell is a cell in the retinal pigment epithelium (RPE). In some embodiments, a cell possesses one or more characteristics, properties and/or activities of a retinal cell.

In some embodiments, a system is a tissue. In some embodiments, a system comprises a tissue. In some embodiments, a system is an organ. In some embodiments, a system comprises an organ. In some embodiments, a system is an eye or a portion thereof. In some embodiments, a system comprises an eye or a portion thereof. In some embodiments, a system is an organism. In some embodiments, a system comprises an organism. In some embodiments, a system is a subject. In some embodiments, a system is a mammal, e.g., a mouse, rat, monkey, or human. In some embodiments, a system is a human.

In some embodiments, a level is increased by about 1 fold, 2 fold, 3 fold, 4 fold, 5 fold, 6 fold, 7 fold, 8 fold, 9 fold, or more as compared to absence of a provided isolated nucleic acid, vector, rAAV, or composition thereof and/or presence of a control (e.g., vehicle only). In some embodiments, a level is increased by about 10%, 20%, 30%, 40%, 50%, 60%, 70%, 80%, 90%, 100%, 150%, 200%, 250%, 300%, 350%, 400%, 450%, 500%, or more as compared to absence of a provided isolated nucleic acid, vector, rAAV, or composition thereof and/or presence of a control (e.g., vehicle only).

In some embodiments, the present disclosure provides a cell comprising an isolated nucleic acid, vector, or rAAV as provided herein. In some embodiments, the present disclosure provides methods of administering or delivering to a cell an isolated nucleic acid, vector, rAAV, or composition thereof as provided herein. In some embodiments, the present disclosure provides methods of increasing expression of PRPF31 in a cell, comprising administering or delivering to the cell an isolated nucleic acid, vector, rAAV, or composition thereof as provided herein. In some embodiments, the present disclosure provides methods of increasing expression of PRPF31 in a cell, comprising administering or delivering to the cell a therapeutically effective amount of an isolated nucleic acid, vector, rAAV, or composition thereof as provided herein. In some embodiments, the present disclosure provides methods of increasing levels of PRPF31 polypeptide in a cell, comprising administering or delivering to the cell an isolated nucleic acid, vector, rAAV, or composition thereof as provided herein. In some embodiments, the present disclosure provides methods of increasing levels of PRPF31 polypeptide in a cell, comprising administering or delivering to the cell a therapeutically effective amount of an isolated nucleic acid, vector, rAAV, or composition thereof as provided herein. In some embodiments, a cell is an isolated cell. In some embodiments, an isolated cell is a prokaryotic cell. In some embodiments, an isolated cell is a eukaryotic cell. In some embodiments, an isolated cell is a mammalian cell. In some embodiments, an isolated cell is an induced pluripotent stem cell (iPSC). In some embodiments, an isolated cell is an iPSC-derived cell. In some embodiments, an isolated cell is a retinal cell. In some embodiments, an isolated cell is a retinal pigment epithelium (RPE) cell. In some embodiments, a cell is a host cell. In some embodiments, a host cell is a prokaryotic cell. In some embodiments, a host cell is a eukaryotic cell. In some embodiments, a host cell is a mammalian cell. In some embodiments, a host cell is a human cell. In some embodiments, a host cell is a retinal cell. In some embodiments, a host cell is a RPE cell.

In some embodiments, the present disclosure provides methods of administering to a subject an isolated nucleic acid, vector, rAAV or composition thereof as provided herein. In some embodiments, the present disclosure provides methods of administering to a subject a therapeutically effective amount of an isolated nucleic acid, vector, rAAV, or composition thereof as provided herein. In some embodiments, an isolated nucleic acid of the present disclosure is administered or delivered via a vector. In some embodiments, an isolated nucleic acid or vector of the present disclosure is administered or delivered via a rAAV.

In some embodiments, the present disclosure provides methods of increasing expression of PRPF31 in a subject, comprising administering an isolated nucleic acid, vector, rAAV, or composition thereof provided herein. In some embodiments, the present disclosure provides methods of increasing expression of PRPF31 in a subject, comprising administering a therapeutically effective amount of an isolated nucleic acid, vector, rAAV, or composition thereof provided herein.

In some embodiments, the present disclosure provides methods of increasing levels of PRPF31 polypeptide in a subject, comprising administering an isolated nucleic acid, vector, rAAV, or composition thereof provided herein. In some embodiments, the present disclosure provides methods of increasing levels of PRPF31 polypeptide in a subject, comprising a therapeutically effective amount of an isolated nucleic acid, vector, rAAV, or composition thereof provided herein.

In some embodiments, the present disclosure provides methods of treating a subject with a disease, disorder, or condition, comprising administering an isolated nucleic acid, vectors, rAAVs, or composition thereof provided herein. In some embodiments, the present disclosure provides methods of treating a subject with a disease, disorder, or condition, comprising administering a therapeutically effective amount of an isolated nucleic acid, vectors, rAAVs, or composition thereof provided herein.

Various diseases, disorders, or conditions associated with PRPF31 may be prevented or treated with provided technologies. In some embodiments, a subject benefits from increased levels of wild-type PRPF31 transcripts, polypeptides, and/or activities in certain cells, tissues, and/or organs. In some embodiments, a disease, disorder, or condition is retinal degeneration. In some embodiments, a disease, disorder, or condition is retinitis pigmentosa (RP). In some embodiments, a disease, disorder, or condition is retinitis pigmentosa-11 (RP 11). In some embodiments, a disease, disorder, or condition is associated with PRPF31. In some embodiments, a disease, disorder, or condition is associated with a mutation in PRPF31.

In some embodiments, an isolated nucleic acid, vector, rAAV, or composition thereof provided herein may be utilized in combination with another therapy, e.g., another therapeutic agent. In some embodiments, an isolated nucleic acid, vector, rAAV, or composition thereof provided herein may be utilized in combination with one or more immunosuppressants. Various immunosuppressants are known in the art. In some embodiments, an immunosuppressant is a steroid. In some embodiments, an immunosuppressant is a corticosteroid. In some embodiments, an immunosuppressant may be an inhibitor of Janus Kinase (JAK). In some embodiments, an immunosuppressant may be a calcineurin inhibitor. In some embodiments, an immunosuppressant may be a cysteine proteinase. In some embodiments, an immunosuppressant may be an antibody or fragment thereof. In some embodiments, an immunosuppressant may be a proteasome inhibitor. In some embodiments, an immunosuppressant is abrocitinib, baricitinib, cyclosporine, dexamethasone (dex), intravenous immune globulin (IVIG), methylprednisolone, mycophenolate mofetil (MMF), mycophenolate sodium, prednisone, rituximab, ruxolitinib, sirolimus (rapamycin), tacrolimus (tacro; Tac), tofacitinib (tofa; TFB), hydroxychloroquine, rabbit anti-thymocyte globulin (rATG), imlifidase, or upadacitinib.

In some embodiments, one or more immunosuppressants may be administered or delivered before administration of an isolated nucleic acid, vector, rAAV, or composition thereof. In some embodiments, the period of time between administration of one or more immunosuppressants and administration of an isolated nucleic acid, vector, rAAV, or composition thereof provided herein may be at least 1 day, at least 2 days, at least 3 days, at least 4 days, at least 5 days, at least 6 days, at least 1 week, at least 2 weeks, at least 3 weeks, at least 1 month, at least 2 months, at least 3 months, at least 6 months, or at least 1 year or more. In some embodiments, one or more immunosuppressants may be administered in multiple doses before administration of an isolated nucleic acid, vector, rAAV, or composition thereof provided herein. In some embodiments, one or more immunosuppressants may be administered for a period of at least 1 day, at least 2 days, at least 3 days, at least 4 days, at least 5 days, at least 6 days, at least 1 week, at least 2 weeks, at least 1 month, at least 2 months, at least 3 months, at least 6 months, or at least 1 year before administration of an isolated nucleic acid, vector, rAAV, or composition thereof provided herein. In some embodiments, one or more immunosuppressants may be administered or delivered concurrently to administration of an isolated nucleic acid, vector, rAAV, or composition thereof provided herein. In some embodiments, one or more immunosuppressants may be administered or delivered following administration of an isolated nucleic acid, vector, rAAV, or composition thereof provided herein. In some embodiments, the period of time between administration of an isolated nucleic acid, vector, rAAV, or composition thereof provided herein and one or more immunosuppressants may be at least 1 day, at least 2 days, at least 3 days, at least 4 days, at least 5 days, at least 6 days, at least 1 week, at least 2 weeks, at least 3 weeks, at least 1 month, at least 2 months, at least 3 months, at least 6 months, or at least 1 year or more. In some embodiments, one or more immunosuppressants may be administered in multiple doses following administration of an isolated nucleic acid, vector, rAAV, or composition thereof provided herein. In some embodiments, one or more immunosuppressants may be administered for a period of at least 1 day, at least 2 days, at least 3 days, at least 4 days, at least 5 days, at least 6 days, at least 1 week, at least 2 weeks, at least 1 month, at least 2 months, at least 3 months, at least 6 months, or at least 1 year following administration of an isolated nucleic acid, vector, rAAV, or composition thereof provided herein. In some embodiments, one or more immunosuppressants may be administered before and following administration of an isolated nucleic acid, vector, rAAV, or composition thereof provided herein. In some embodiments, one or more immunosuppressants may be administered before and concurrently with administration of an isolated nucleic acid, vector, rAAV, or composition thereof provided herein. In some embodiments, one or more immunosuppressants may be administered concurrently with and following administration of an isolated nucleic acid, vector, rAAV, or composition thereof provided herein. In some embodiments, one or more immunosuppressants may be administered before, concurrently with, and following administration of an isolated nucleic acid, vector, rAAV, or composition thereof provided herein.

In some embodiments, provided technologies (e.g., isolated nucleic acids, vectors, rAAVs, compositions, methods, etc.) delay or prevent onset of one or more symptoms and/or hallmarks of a disease, disorder, or condition. In some embodiments, provided technologies delay, slow down, or prevent progression of a disease, disorder, or condition. In some embodiments, provided technologies alleviate, ameliorate, relieve, inhibit, prevent, delay onset of reduce severity of, and/or reduce incidence of one or more symptoms or features of a disease, disorder, or condition. In some embodiments, provided technologies improve performance of a subject in one or more clinical assessments. In some embodiments, provided technologies independently improve one or more clinical assessment results of a subject. Pharmaceutical Compositions

In general, compositions of the present disclosure may be administered in any form, including tablet, powder, or liquid, formulated into a pharmaceutically acceptable carrier or excipient, depending on the condition of the patient. Additionally, non-active ingredients well known in the art, such as binders, fillers, coatings, preservatives, coloring agents, flavoring agents and other additives may optionally be formulated with one or more administered agents, or left out completely if there is a risk of negative side effects to the patient such as increased the risk of intestinal inflammation or interference with the absorption of particular compounds.

In some embodiments, the present disclosure provides pharmaceutical compositions comprising a provided composition, e.g., an isolated nucleic acid, vector, or rAAV. In some embodiments, for example, for therapeutic and clinical purposes, an isolated nucleic acid, vector, or rAAV are provided as pharmaceutical compositions.

In some embodiments, a pharmaceutical composition is suitable for administration or delivery of an isolated nucleic acid, vector, or rAAV, to an area or portion of a body affected by a disease, disorder, or condition. In some embodiments, a pharmaceutical composition comprises a therapeutically effective amount of an isolated nucleic acid, vector, or rAAV provided herein. In some embodiments, a pharmaceutical composition comprises a therapeutically effective amount of an isolated nucleic acid, vector, or rAAV, and a pharmaceutically acceptable carrier or excipient. In some embodiments, a pharmaceutically acceptable carrier is a buffer.

In some embodiments, a pharmaceutical composition is formulated for intravenous injection, oral administration, buccal administration, inhalation, nasal administration, topical administration, ophthalmic administration, or otic administration. In some embodiments, a pharmaceutical composition is a tablet, a pill, a capsule, a liquid, an inhalant, a nasal spray solution, a suppository, a suspension, a gel, a colloid, a dispersion, a suspension, a solution, an emulsion, an ointment, a lotion, an eye drop, or an ear drop. In some embodiments, a pharmaceutical composition is formulated for subretinal administration.

Various technologies may be utilized to administer or deliver provided an isolated nucleic acids, vectors, rAAVs, or compositions thereof. In some embodiments, provided isolated nucleic acids, vectors, or rAAVs, or compositions thereof are delivered to the eye. In some embodiments, provided isolated nucleic acids, vectors, or rAAVs, or compositions thereof are delivered to the subretinal space. In some embodiments, provided isolated nucleic acids, vectors, or rAAVs, or compositions thereof are delivered to a subject by subretinal administration. In some embodiments, subretinal administration is by injection, by, e.g., a syringe. In some embodiments, an injection is a bolus injection.

Provided isolated nucleic acids, vectors, and rAAVs, and compositions thereof can be administered over a wide dose range. In some embodiments, a vector or rAAV, or composition thereof is administered to a subject at a dose of at least 108, at least 10⁹, at least 10¹⁰, at least 10¹, at least 10¹², at least 10¹³, at least 10¹⁴, at least 10¹⁵, at least 10¹⁶, at least 10¹⁷, at least 10¹⁸, at least 10¹⁹, at least 10²⁰, at least 10²¹, at least 10²², at least 10²³, at least 10²⁴, at least 10²⁵, or at least 10²⁶ genome copies per subject. In some embodiments, a vector or rAAV, or composition thereof is administered to a subject at a dose of at most 10⁸, at most 10⁹, at most 10¹⁰, at most 10¹¹, at most 10¹², at most 10¹³, at most 10¹⁴, at most 10¹⁵, at most 10¹⁶, at most 10¹⁷, at most 10¹⁸, at most 10¹⁹, at most 10²⁰, at most 10²¹, at most 10²², at most 10²³, at most 10²⁴, at most 10²¹, or at most 10²⁶ genome copies per subject. In some embodiments, a vector or rAAV, or composition thereof is administered to a subject at a dose of at least 108, at least 10⁹, at least 10¹⁰, at least 10¹¹, at least 10¹², at least 10¹³, or at least 1014 genome copies per eye. In some embodiments, a vector or rAAV, or composition thereof is administered to a subject at a dose of at most 10⁸, at most 10⁹, at most 10¹⁰, at most 10¹¹, at most 10¹², at most 10¹³, or at most 10¹⁴ genome copies per eye. In some embodiments, a vector or rAAV, or composition thereof is administered to a subject at a dose within a range of about 10⁸ to about 10²⁶, 10¹⁰ to about 10²⁴, or 10¹² to about 10²² genome copies per subject. In some embodiments, a vector or rAAV, or composition thereof is administered to a subject at a dose within a range of about 10⁸ to about 10¹⁴ genome copies, about 10⁹ to about 10¹³ genome copies, or about 10¹⁰ to about 10¹² genome copies per eye. In some embodiments, a vector or rAAV, or composition thereof is administered to a subject at a dose of about 10⁹ genome copies per eye. In some embodiments, a vector or rAAV, or composition thereof is administered to a subject at a dose of about 5×10⁹ genomes copies per eye. In some embodiments, a vector or rAAV, or composition thereof is administered to a subject at a dose of about 10¹⁰ genome copies per eye. In some embodiments, a vector or rAAV, or composition thereof is administered to a subject at a dose of about 5×10¹⁰ genomes copies per eye. In some embodiments, a vector or rAAV, or composition thereof is administered to a subject at a dose of about 10¹¹ genome copies per eye. In some embodiments, a vector or rAAV, or composition thereof is administered to a subject at a dose of about 5×10¹¹ genome copies per eye. In some embodiments, a vector or rAAV, or composition thereof is administered to a subject at a dose of about 10¹² genome copies per eye.

### Sequences

Various nucleic acid sequences may be used in the provided technologies of the present disclosure. Exemplary sequences are provided below.

-Human PRPF31 cDNA
SEQ ID NO: 1
ATGTCTCTGGCAGATGAGCTCTTAGCTGATCTCGAAGAGGCAGCAGAAGAGGAGGAAGGAGGAAGCTATG
GGGAGGAAGAAGAGGAGCCAGCGATCGAGGATGTGCAGGAGGAGACACAGCTGGATCTTTCCGGGGATTC
AGTCAAGACCATCGCCAAGCTATGGGATAGTAAGATGTTTGCTGAGATTATGATGAAGATTGAGGAGTAT
ATCAGCAAGCAAGCCAAAGCTTCAGAAGTGATGGGACCAGTGGAGGCCGCGCCTGAATACCGCGTCATCG
TGGATGCCAACAACCTGACCGTGGAGATCGAAAACGAGCTGAACATCATCCATAAGTTCATCCGGGATAA
GTACTCAAAGAGATTCCCTGAACTGGAGTCCTTGGTCCCCAATGCACTGGATTACATCCGCACGGTCAAG
GAGCTGGGCAACAGCCTGGACAAGTGCAAGAACAATGAGAACCTGCAGCAGATCCTCACCAATGCCACCA
TCATGGTCGTCAGCGTCACCGCCTCCACCACCCAGGGGCAGCAGCTGTCGGAGGAGGAGCTGGAGCGGCT
GGAGGAGGCCTGCGACATGGCGCTGGAGCTGAACGCCTCCAAGCACCGCATCTACGAGTATGTGGAGTCC
CGGATGTCCTTCATCGCACCCAACCTGTCCATCATTATCGGGGCATCCACGGCCGCCAAGATCATGGGTG
TGGCCGGCGGCCTGACCAACCTCTCCAAGATGCCCGCCTGCAACATCATGCTGCTCGGGGCCCAGCGCAA
GACGCTGTCGGGCTTCTCGTCTACCTCAGTGCTGCCCCACACCGGCTACATCTACCACAGTGACATCGTG
CAGTCCCTGCCACCGGATCTGCGGCGGAAAGCGGCCCGGCTGGTGGCCGCCAAGTGCACACTGGCAGCCC
GTGTGGACAGTTTCCACGAGAGCACAGAAGGGAAGGTGGGCTACGAACTGAAGGATGAGATCGAGCGCAA
ATTCGACAAGTGGCAGGAGCCGCCGCCTGTGAAGCAGGTGAAGCCGCTGCCTGCGCCCCTGGATGGACAG
CGGAAGAAGCGAGGCGGCCGCAGGTACCGCAAGATGAAGGAGCGGCTGGGGCTGACGGAGATCCGGAAGC
AGGCCAACCGTATGAGCTTCGGAGAGATCGAGGAGGACGCCTACCAGGAGGACCTGGGATTCAGCCTGGG
CCACCTGGGCAAGTCGGGCAGTGGGCGTGTGCGGCAGACACAGGTAAACGAGGCCACCAAGGCCAGGATC
TCCAAGACGCTGCAGCGGACCCTGCAGAAGCAGAGCGTCGTATATGGCGGGAAGTCCACCATCCGCGACC
GCTCCTCGGGCACGGCCTCCAGCGTGGCCTTCACCCCACTCCAGGGCCTGGAGATTGTGAACCCACAGGC
GGCAGAGAAGAAGGTGGCTGAGGCCAACCAGAAGTATTTCTCCAGCATGGCTGAGTTCCTCAAGGTCAAG
GGCGAGAAGAGTGGCCTTATGTCCACCTGA
-AAV2 5′ ITR
SEQ ID NO: 2
CCTGCAGGCAGCTGCGCGCTCGCTCGCTCACTGAGGCCGCCCGGGCGTCGGGCGACCTTTGGTCGCCCGG
CCTCAGTGAGCGAGCGAGCGCGCAGAGAGGGAGTGGCCAACTCCATCACTAGGGGTTCCT
-AAV2 3′ ITR
SEQ ID NO: 3
AGGAACCCCTAGTGATGGAGTTGGCCACTCCCTCTCTGCGCGCTCGCTCGCTCACTGAGGCCGGGCGACC
AAAGGTCGCCCGACGCCCGGGCTTTGCCCGGGCGGCCTCAGTGAGCGAGCGAGCGCGCAGCTGCCTGCAG
G
-Promoter
SEQ ID NO: 4
GACATTGATTATTGACTAGTTATTAATAGTAATCAATTACGGGGTCATTAGTTCATAGCCCATATATGGA
GTTCCGCGTTACATAACTTACGGTAAATGGCCCGCCTGGCTGACCGCCCAACGACCCCCGCCCATTGACG
TCAATAATGACGTATGTTCCCATAGTAACGCCAATAGGGACTTTCCATTGACGTCAATGGGTGGAGTATT
TACGGTAAACTGCCCACTTGGCAGTACATCAAGTGTATCATATGCCAAGTACGCCCCCTATTGACGTCAA
TGACGGTAAATGGCCCGCCTGGCATTATGCCCAGTACATGACCTTATGGGACTTTCCTACTTGGCAGTAC
ATCTACGTATTAGTCATCGCTATTACCATGGTCGAGGTGAGCCCCACGTTCTGCTTCACTCTCCCCATCT
CCCCCCCCTCCCCACCCCCAATTTTGTATTTATTTATTTTTTAATTATTTTGTGCAGCGATGGGGGCGGG
GGGGGGGGGGGGGCGCGCGCCAGGCGGGGCGGGGCGGGGCGAGGGGCGGGGCGGGGCGAGGCGGAGAGGT
GCGGCGGCAGCCAATCAGAGCGGCGCGCTCCGAAAGTTTCCTTTTATGGCGAGGCGGCGGCGGCGGCGGC
CCTATAAAAAGCGAAGCGCGCGGCGGGCGGGAGTCGCTGCGCGCTGCCTTCGCCCCGTGCCCCGCTCCGC
CGCCGCCTCGCGCCGCCCGCCCCGGCTCTGACTGACCGCGTTACTAAAACAGGTAAGTCCGGCCTCCGCG
CCGGGTTTTGGCGCCTCCCGCGGGCGCCCCCCTCCTCACGGCGAGCGCTGCCACGTCAGACGAAGGGCGC
AGCGAGCGTCCTGATCCTTCCGCCCGGACGCTCAGGACAGCGGCCCGCTGCTCATAAGACTCGGCCTTAG
AACCCCAGTATCAGCAGAAGGACATTTTAGGACGGGACTTGGGTGACTCTAGGGCACTGGTTTTCTTTCC
AGAGAGCGGAACAGGCGAGGAAAAGTAGTCCCTTCTCGGCGATTCTGCGGAGGGATCTCCGTGGGGCGGT
GAACGCCGATGATGCCTCTACTAACCATGTTCATGTTTTCTTTTTTTTTCTACAGGTCCTGGGTGACGAA
CAG
-WPRE
SEQ ID NO: 5
CAATCAACCTCTGGATTACAAAATTTGTGAAAGATTGACTGGTATTCTTAACTATGTTGCTCCTTTTACG
CTATGTGGATACGCTGCTTTAATGCCTTTGTATCATGCTATTGCTTCCCGTATGGCTTTCATTTTCTCCT
CCTTGTATAAATCCTGGTTGCTGTCTCTTTATGAGGAGTTGTGGCCCGTTGTCAGGCAACGTGGCGTGGT
GTGCACTGTGTTTGCTGACGCAACCCCCACTGGTTGGGGCATTGCCACCACCTGTCAGCTCCTTTCCGGG
ACTTTCGCTTTCCCCCTCCCTATTGCCACGGCGGAACTCATCGCCGCCTGCCTTGCCCGCTGCTGGACAG
GGGCTCGGCTGTTGGGCACTGACAATTCCGTGGTGTTGTCGGGGAAATCATCGTCCTTTCCTTGGCTGCT
CGCCTGTGTTGCCACCTGGATTCTGCGCGGGACGTCCTTCTGCTACGTCCCTTCGGCCCTCAATCCAGCG
GACCTTCCTTCCCGCGGCCTGCTGCCGGCTCTGCGGCCTCTTCCGCGTCTTCGCCTTCGCCCTCAGACGA
GTCGGATCTCCCTTTGGGCCGCCTCCCCGC
-Human Beta-Globin Polyadenylation Signal
SEQ ID NO: 6
GCTCGCTTTCTTGCTGTCCAATTTCTATTAAAGGTTCCTTTGTTCCCTAAGTCCAACTACTAAACTGGGG
GATATTATGAAGGGCCTTGAGCATCTGGATTCTGCCTAATAAAAAACATTTATTTTCATTGCAATGATGT
ATTTAAATTATTTCTGAATATTTTACTAAAAAGGGAATGTGGGAGGTCAGTGCATTTAAAACATAAAGAA
ATGAAGAGCTAGTTCAAACCTTGGGAAAATACACTATATCTTAAACTCCATGAAAGAAGGTGAGGCTGCA
AACAGCTAATGCACATTGGCAACAGCCCCTGATGCATATGCCTTATTCATCCCTCAGAAAAGGATTCAAG
TAGAGGCTTGATTTGGAGGTTAAAGTTTTGCTATGCTGTATTTTA
-Exemplary AAV Vector
SEQ ID NO: 7
CCTGCAGGCAGCTGCGCGCTCGCTCGCTCACTGAGGCCGCCCGGGCGTCGGGCGACCTTTGGTCGCCCGG
CCTCAGTGAGCGAGCGAGCGCGCAGAGAGGGAGTGGCCAACTCCATCACTAGGGGTTCCTGCGGCCGGTC
GACATTGATTATTGACTAGTTATTAATAGTAATCAATTACGGGGTCATTAGTTCATAGCCCATATATGGA
GTTCCGCGTTACATAACTTACGGTAAATGGCCCGCCTGGCTGACCGCCCAACGACCCCCGCCCATTGACG
TCAATAATGACGTATGTTCCCATAGTAACGCCAATAGGGACTTTCCATTGACGTCAATGGGTGGAGTATT
TACGGTAAACTGCCCACTTGGCAGTACATCAAGTGTATCATATGCCAAGTACGCCCCCTATTGACGTCAA
TGACGGTAAATGGCCCGCCTGGCATTATGCCCAGTACATGACCTTATGGGACTTTCCTACTTGGCAGTAC
ATCTACGTATTAGTCATCGCTATTACCATGGTCGAGGTGAGCCCCACGTTCTGCTTCACTCTCCCCATCT
CCCCCCCCTCCCCACCCCCAATTTTGTATTTATTTATTTTTTAATTATTTTGTGCAGCGATGGGGGCGGG
GGGGGGGGGGGGGCGCGCGCCAGGCGGGGCGGGGCGGGGCGAGGGGCGGGGCGGGGCGAGGCGGAGAGGT
GCGGCGGCAGCCAATCAGAGCGGCGCGCTCCGAAAGTTTCCTTTTATGGCGAGGCGGCGGCGGCGGCGGC
CCTATAAAAAGCGAAGCGCGCGGCGGGCGGGAGTCGCTGCGCGCTGCCTTCGCCCCGTGCCCCGCTCCGC
CGCCGCCTCGCGCCGCCCGCCCCGGCTCTGACTGACCGCGTTACTAAAACAGGTAAGTCCGGCCTCCGCG
CCGGGTTTTGGCGCCTCCCGCGGGCGCCCCCCTCCTCACGGCGAGCGCTGCCACGTCAGACGAAGGGCGC
AGCGAGCGTCCTGATCCTTCCGCCCGGACGCTCAGGACAGCGGCCCGCTGCTCATAAGACTCGGCCTTAG
AACCCCAGTATCAGCAGAAGGACATTTTAGGACGGGACTTGGGTGACTCTAGGGCACTGGTTTTCTTTCC
AGAGAGCGGAACAGGCGAGGAAAAGTAGTCCCTTCTCGGCGATTCTGCGGAGGGATCTCCGTGGGGCGGT
GAACGCCGATGATGCCTCTACTAACCATGTTCATGTTTTCTTTTTTTTTCTACAGGTCCTGGGTGACGAA
CAGGCCACCATGTCTCTGGCAGATGAGCTCTTAGCTGATCTCGAAGAGGCAGCAGAAGAGGAGGAAGGAG
GAAGCTATGGGGAGGAAGAAGAGGAGCCAGCGATCGAGGATGTGCAGGAGGAGACACAGCTGGATCTTTC
CGGGGATTCAGTCAAGACCATCGCCAAGCTATGGGATAGTAAGATGTTTGCTGAGATTATGATGAAGATT
GAGGAGTATATCAGCAAGCAAGCCAAAGCTTCAGAAGTGATGGGACCAGTGGAGGCCGCGCCTGAATACC
GCGTCATCGTGGATGCCAACAACCTGACCGTGGAGATCGAAAACGAGCTGAACATCATCCATAAGTTCAT
CCGGGATAAGTACTCAAAGAGATTCCCTGAACTGGAGTCCTTGGTCCCCAATGCACTGGATTACATCCGC
ACGGTCAAGGAGCTGGGCAACAGCCTGGACAAGTGCAAGAACAATGAGAACCTGCAGCAGATCCTCACCA
ATGCCACCATCATGGTCGTCAGCGTCACCGCCTCCACCACCCAGGGGCAGCAGCTGTCGGAGGAGGAGCT
GGAGCGGCTGGAGGAGGCCTGCGACATGGCGCTGGAGCTGAACGCCTCCAAGCACCGCATCTACGAGTAT
GTGGAGTCCCGGATGTCCTTCATCGCACCCAACCTGTCCATCATTATCGGGGCATCCACGGCCGCCAAGA
TCATGGGTGTGGCCGGCGGCCTGACCAACCTCTCCAAGATGCCCGCCTGCAACATCATGCTGCTCGGGGC
CCAGCGCAAGACGCTGTCGGGCTTCTCGTCTACCTCAGTGCTGCCCCACACCGGCTACATCTACCACAGT
GACATCGTGCAGTCCCTGCCACCGGATCTGCGGCGGAAAGCGGCCCGGCTGGTGGCCGCCAAGTGCACAC
TGGCAGCCCGTGTGGACAGTTTCCACGAGAGCACAGAAGGGAAGGTGGGCTACGAACTGAAGGATGAGAT
CGAGCGCAAATTCGACAAGTGGCAGGAGCCGCCGCCTGTGAAGCAGGTGAAGCCGCTGCCTGCGCCCCTG
GATGGACAGCGGAAGAAGCGAGGCGGCCGCAGGTACCGCAAGATGAAGGAGCGGCTGGGGCTGACGGAGA
TCCGGAAGCAGGCCAACCGTATGAGCTTCGGAGAGATCGAGGAGGACGCCTACCAGGAGGACCTGGGATT
CAGCCTGGGCCACCTGGGCAAGTCGGGCAGTGGGCGTGTGCGGCAGACACAGGTAAACGAGGCCACCAAG
GCCAGGATCTCCAAGACGCTGCAGCGGACCCTGCAGAAGCAGAGCGTCGTATATGGCGGGAAGTCCACCA
TCCGCGACCGCTCCTCGGGCACGGCCTCCAGCGTGGCCTTCACCCCACTCCAGGGCCTGGAGATTGTGAA
CCCACAGGCGGCAGAGAAGAAGGTGGCTGAGGCCAACCAGAAGTATTTCTCCAGCATGGCTGAGTTCCTC
AAGGTCAAGGGCGAGAAGAGTGGCCTTATGTCCACCTGACAATCAACCTCTGGATTACAAAATTTGTGAA
AGATTGACTGGTATTCTTAACTATGTTGCTCCTTTTACGCTATGTGGATACGCTGCTTTAATGCCTTTGT
ATCATGCTATTGCTTCCCGTATGGCTTTCATTTTCTCCTCCTTGTATAAATCCTGGTTGCTGTCTCTTTA
TGAGGAGTTGTGGCCCGTTGTCAGGCAACGTGGCGTGGTGTGCACTGTGTTTGCTGACGCAACCCCCACT
GGTTGGGGCATTGCCACCACCTGTCAGCTCCTTTCCGGGACTTTCGCTTTCCCCCTCCCTATTGCCACGG
CGGAACTCATCGCCGCCTGCCTTGCCCGCTGCTGGACAGGGGCTCGGCTGTTGGGCACTGACAATTCCGT
GGTGTTGTCGGGGAAATCATCGTCCTTTCCTTGGCTGCTCGCCTGTGTTGCCACCTGGATTCTGCGCGGG
ACGTCCTTCTGCTACGTCCCTTCGGCCCTCAATCCAGCGGACCTTCCTTCCCGCGGCCTGCTGCCGGCTC
TGCGGCCTCTTCCGCGTCTTCGCCTTCGCCCTCAGACGAGTCGGATCTCCCTTTGGGCCGCCTCCCCGCG
CTCGCTTTCTTGCTGTCCAATTTCTATTAAAGGTTCCTTTGTTCCCTAAGTCCAACTACTAAACTGGGGG
ATATTATGAAGGGCCTTGAGCATCTGGATTCTGCCTAATAAAAAACATTTATTTTCATTGCAATGATGTA
TTTAAATTATTTCTGAATATTTTACTAAAAAGGGAATGTGGGAGGTCAGTGCATTTAAAACATAAAGAAA
TGAAGAGCTAGTTCAAACCTTGGGAAAATACACTATATCTTAAACTCCATGAAAGAAGGTGAGGCTGCAA
ACAGCTAATGCACATTGGCAACAGCCCCTGATGCATATGCCTTATTCATCCCTCAGAAAAGGATTCAAGT
AGAGGCTTGATTTGGAGGTTAAAGTTTTGCTATGCTGTATTTTAAGGAACCCCTAGTGATGGAGTTGGCC
ACTCCCTCTCTGCGCGCTCGCTCGCTCACTGAGGCCGGGCGACCAAAGGTCGCCCGACGCCCGGGCTTTG
CCCGGGCGGCCTCAGTGAGCGAGCGAGCGCGCAGCTGCCTGCAGG

### EXEMPLIFICATION

### Example 1. Provided Technologies can Provide Robust PRPF31 Expression in Target Cells In Vivo

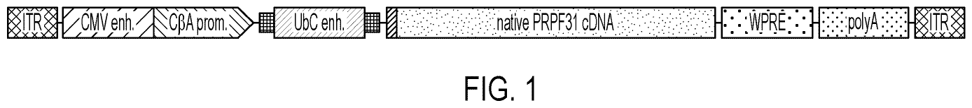

An isolated nucleic acid, similar to as depicted in FIG. 1, was constructed. The isolated nucleic acid comprised, from 5′ to 3′, an AAV2 5′ inverted terminal repeat (ITR) (SEQ ID NO: 2); a promoter (SEQ ID NO: 4) comprising a human cytomegalovirus (CMV) enhancer, a chicken beta-actin (CBA) promoter, a splice donor, a human ubiquitin C (UbC) enhancer, and a splice acceptor; native human PRPF31 cDNA (SEQ ID NO: 1) with a fused N-terminal V5 epitope tag; a woodchuck hepatitis virus post-transcriptional regulatory element (WPRE) (SEQ ID NO: 5); a human beta-globin polyadenylation signal (SEQ ID NO: 6); and an AAV2 3′ ITR (SEQ ID NO: 3). Using the isolated nucleic acid as the basis for an AAV vector, a recombinant AAV (rAAV) was assembled using a triple transfection method as described herein and as known in the art. The rAAV comprising the vector comprising the isolated nucleic acid as described above was termed AAV-PRPF31.

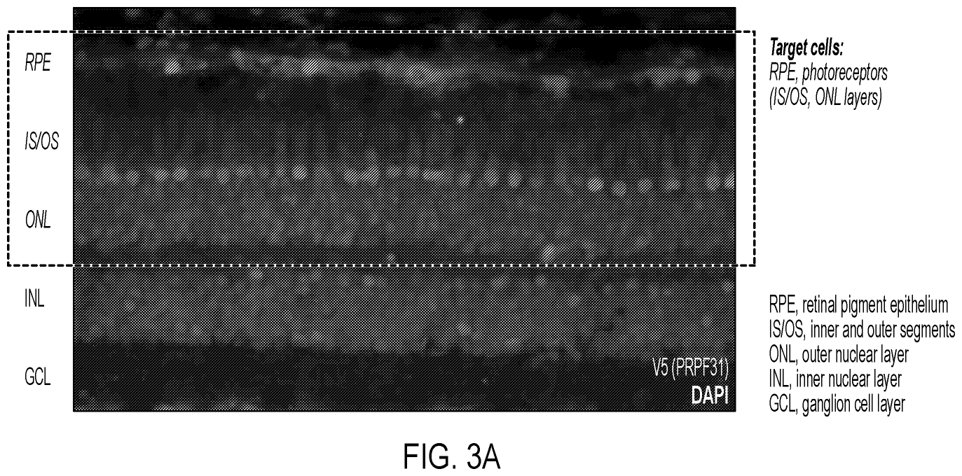

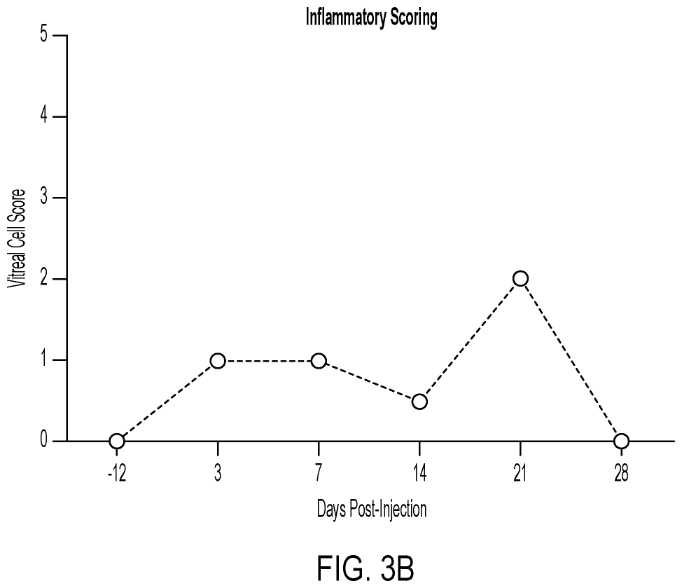

Non-human primates (cynomolgus macaques) received subretinal injections of AAV-PRPF31 at a dose of 5×10¹¹ vg/eye. Animals were treated with methylprednisolone on the day before administration of the rAAV and once per week thereafter. The successful formation of the subretinal bleb in animals was observed by fundus photography (data not shown). Further, inflammation was assessed on day 12 before administration and days 3, 7, 14, 21, and 28 following administration. Inflammation was assessed using uveitis scoring (0=none, 1=minimal, 2=mild, 3=moderate, 4=marked, 5=severe). As shown in FIG. 3B, minimal inflammation was observed across all time points—indicating that administration of an immunosuppressive agent before and following administration of a rAAV as described herein (e.g., AAV-PRPF31) may provide decreased inflammation in a subject.

At 28 days following administration, animals were euthanized and eyes were harvested, fixed, and processed as retinal cross sections for histological analysis as known in the art. Retinal cross sections were immunolabeled with antibodies against the V5 epitope tag (to identify vector-borne PRPF31) and DAPI. Imaging revealed that robust vector-borne PRPF31 expression was present throughout the retinal pigment epithelium (RPE), photoreceptor layers (e.g., inner and outer segments), and inner retina (e.g., outer nuclear layer) (FIG. 3A). These results indicate that a rAAV as described herein (e.g., AAV-PRPF31) may provide robust PRPF31 expression in vivo, e.g., in the target cells of the retina.

### Example 2. Provided Technologies can Provide Increased PRPF31 Expression in Target Cells In Vivo

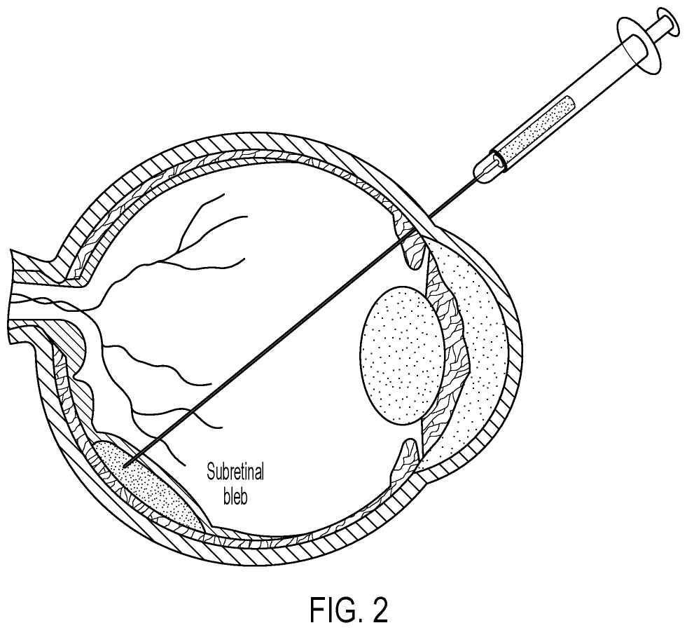

Mutant (Prpf31^{+/-}) mice received subretinal injections of 2×10⁹ vg/eye of AAV-PRPF31 (assembled as described in Example 1) or a vehicle control. Wild-type (Prpf31^{+/+}) mice which did not receive an injection were also examined. At 13 weeks following injection, animals were sacrificed and retinal tissues were collected to examine vector-borne PRPF31 expression in the retinal pigment epithelium (RPE). Retinal cross-sections were immunolabeled with antibodies against PRPF31 and V5 and DAPI. For mutant (Prpf31^{+/-}) mice which received AAV-PRPF31, retinal tissue from within the subretinal bleb area (as formed by the subretinal injection as depicted in FIG. 2) and from outside the subretinal bleb area were examined.

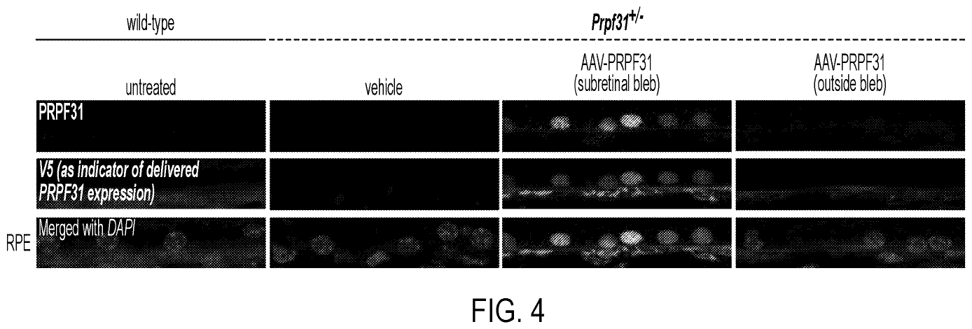

Exemplary micrograph images are shown in FIG. 4. As seen in micrographs from within the subretinal bleb in mutant (Prpf31^{+/-}) mice (third column) substantial vector-borne PRPF31 expression was observed. Meanwhile, substantial PRPF31 expression is not observed outside the subretinal bleb (fourth column), in mutant (Prpf31^{+/-}) mice that received only the vehicle control (second column), or in untreated wild-type (Prpf31^{+/+}) mice (first column). These results indicate that a rAAV as described herein (e.g., AAV-PRPF31) may provide increased PRPF31 expression in vivo, e.g., in the RPE.

### Example 3. Provided Technologies can Provide Increased PRPF31 Expression in Target Cells In Vivo

Mutant (Prpf31^{-/-}) mice received subretinal injections of 2×10⁹ (low dose) or 2×10¹⁰ (high dose) vg/eye of AAV-PRPF31 (assembled as described in Example 1) or a vehicle control. At 13 weeks following injection, animals were sacrificed and eyes were harvested. Posterior eye cups (PECs) containing the retinal pigment epithelium (RPE) layer were dissociated from the neural retina and underwent protein extraction. Protein content was analyzed by western blot. An approximate 54 kDa band associated with PRPF31 was observed and expression of PRPF31 was quantified and normalized to the corresponding expression of GAPDH (for low dose mice) or β-actin (for high dose mice).

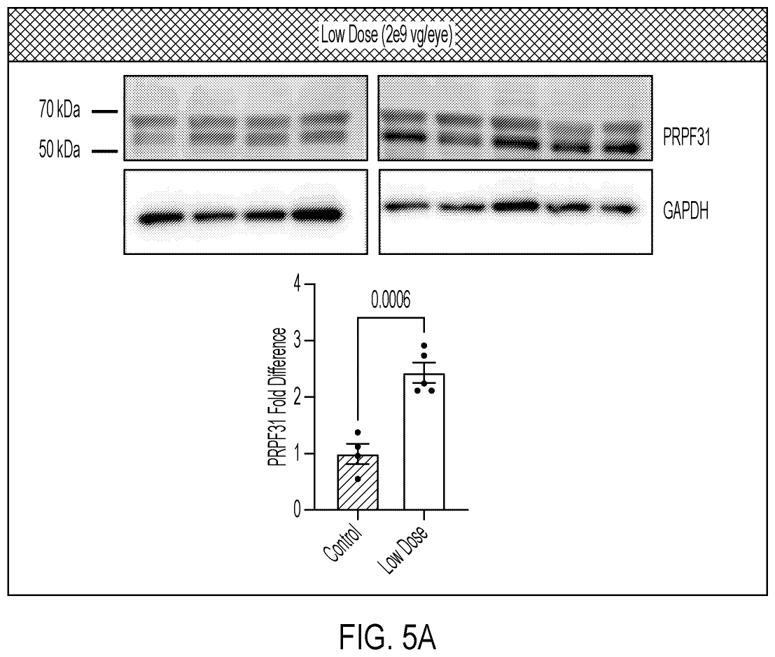

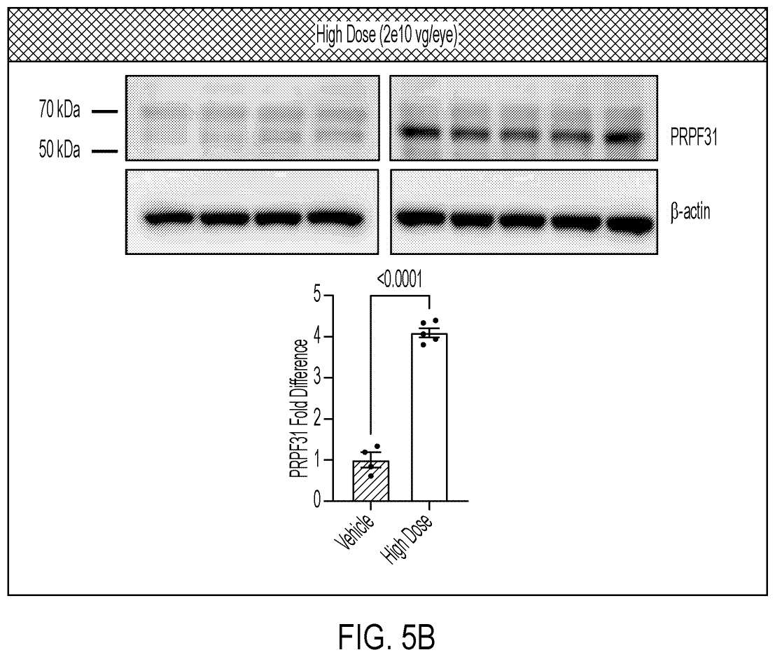

As shown in the graph in FIG. 5A, a significant increase (>2 fold difference) in PRPF31 expression was observed in samples from mice that were injected with the low dose of AAV-PRPF31 as compared to mice that received the vehicle control. Meanwhile, as shown in FIG. 5B, a significant increase (approximately a 4 fold difference) in PRPF31 expression was observed in samples from mice that were injected with the high dose of AAV-PRPF31 as compared to mice that received the vehicle control. These results indicate that a rAAV as described herein (e.g., AAV-PRPF31) may provide increased PRPF31 expression in vivo, e.g., in the RPE, at various doses.

### Example 4. Provided Technologies can Provide Increased Phagocytosis in Target Cells In Vivo

Mutant (Prpf31^{-/-}) mice received subretinal injections of 2×10⁹ (low dose) or 2×10¹⁰ (high dose) vg/eye of AAV-PRPF31 (assembled as described in Example 1) or a vehicle control. Wild-type (Prpf31^{+/+}) mice which did not receive an injection were also examined. At 13 weeks post-injection, animals were sacrificed and retinal tissues were collected to examine phagocytosis in the retinal pigment epithelium (RPE). Retinal cross-sections were immunolabeled with antibodies against PRPF31, F-actin, and rhodopsin (RHO) and DAPI. RHO phagosomes in the RPE layer were quantified per 100 μm sections via microscopy.

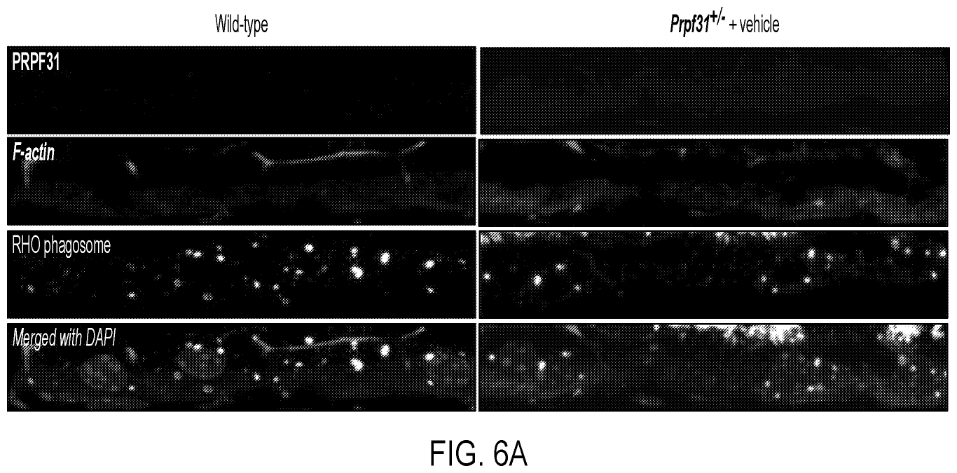

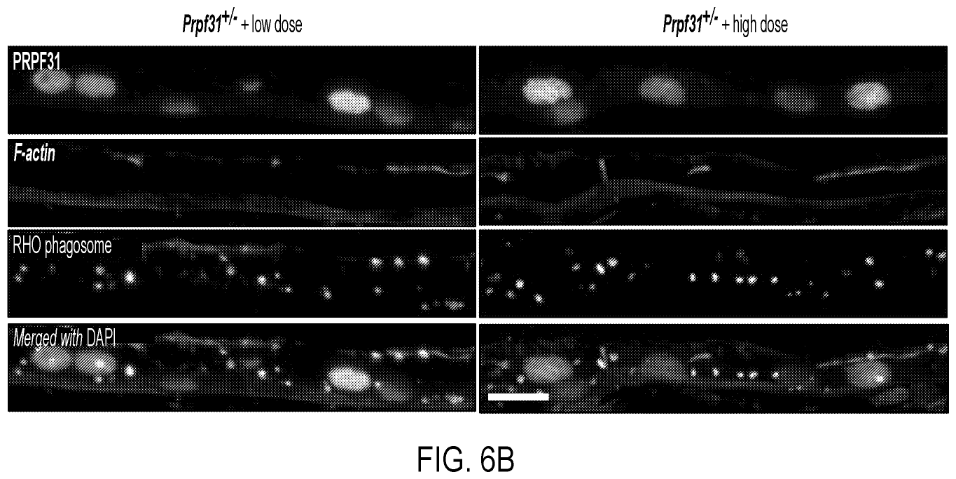

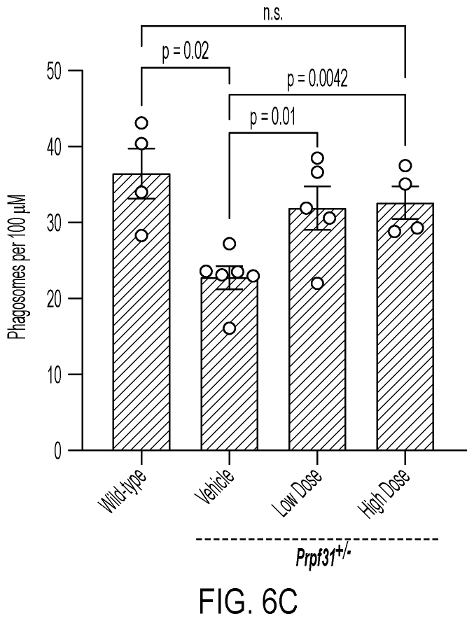

Exemplary micrograph images are shown in FIGS. 6A and 6B. As shown in FIG. 6C, untreated wild-type (Prpf31^{+/+}) mice displayed significantly greater RI-TO phagosomes than vehicle-treated mutant (Prpf31^{+/-}) mice. Further, mutant (Prpf31^{+/-}) mice treated with either a low dose or high dose of AAV-PRPF31 displayed similar quantities of RHO phagosomes as wild-type (Prpf31^{+/+}) mice and a significantly greater number of RHO phagosomes than in mutant (Prpf31^{+/-}) mice treated with vehicle (FIG. 6C. These results indicate that a rAAV as described herein (e.g., AAV-PRPF31) may provide increased phagocytosis in vivo. e.g., in the RPE, at various doses.

### Example 5. Provided Technologies can Provide Increased Phagocytosis in Target Cells In Vitro

Wild-type (Prpf31^{+/+}) and mutant (Prpf31^{-/-}) induced pluripotent stem cell (iPSC)-derived retinal pigment epithelium (RPE) cells were plated and matured for three weeks. AAV-PRPF31 (a rAAV comprising the construct depicted in FIG. 1) was constructed as described in Example 1 and administered to the cultures at a multiplicity of infection (MOI) of 5×105. Cells were cultured for three weeks post-administration before phagocytic capacity was assayed by treatment with fluorescein isothiocyanate (FITC)-conjugated photoreceptor outer segments (POS). Cells were evaluated thereafter by microscopy.

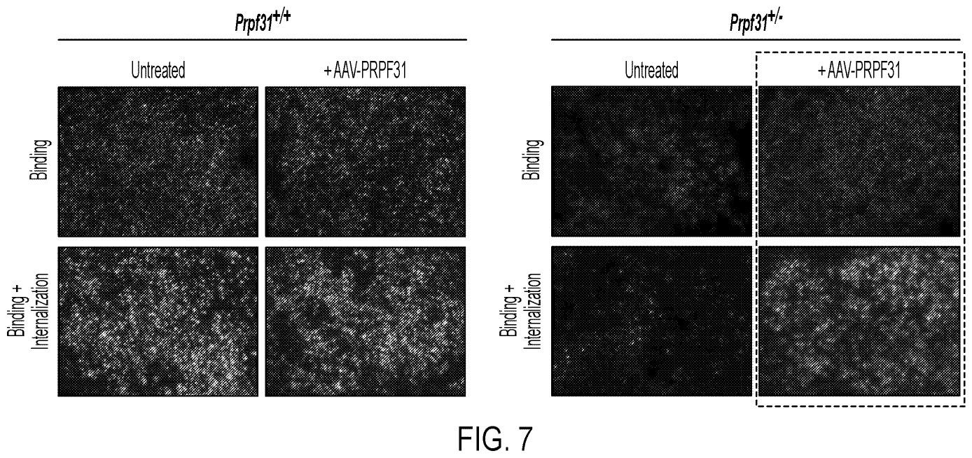

As shown in FIG. 7, mutant (Prpf31^{+/-}) cells display a reduced ability to bind and internalize POS as compared to wild-type (Prpf31^{+/+}) cells as shown in the decreased FITC signal in the untreated column of micrographs (compare first column and third column of micrographs). Meanwhile, treatment of cells with AAV-PRPF31 restored phagocytosis to the cells as demonstrated by the increased FITC signal following binding and internalization of POS (rightmost column of micrographs in FIG. 7). These results indicate that a rAAV as described herein (e.g., AAV-PRPF31) may provide increased phagocytosis in vitro.

While various embodiments have been described and illustrated herein, those of ordinary skill in the art will readily envision a variety of other means and/or structures for performing the functions and/or obtaining the results and/or one or more of the advantages described in the present disclosure, and each of such variations and/or modifications is deemed to be included. More generally, those skilled in the art will readily appreciate that all parameters, dimensions, materials, and configurations described herein are meant to be example and that the actual parameters, dimensions, materials, and/or configurations may depend upon the specific application or applications for which the teachings of the present disclosure is/are used. Those skilled in the art will recognize, or be able to ascertain using no more than routine experimentation, many equivalents to the embodiments of the present disclosure. It is, therefore, to be understood that the foregoing embodiments are presented by way of example only and that, within the scope of the appended claims and equivalents thereto, claimed technologies may be practiced otherwise than as specifically described and claimed. In addition, any combination of two or more features, systems, articles, materials, kits, and/or methods, if such features, systems, articles, materials, kits, and/or methods are not mutually inconsistent, is included within the scope of the present disclosure.

1. An isolated nucleic acid comprising:
(i) an adeno-associated virus (AAV) 5′ inverted terminal repeat (ITR);
(ii) a promoter;
(iii) a nucleic acid sequence encoding a PRPF31 polypeptide;
(iv) a woodchuck hepatitis virus post-transcriptional regulatory element (WPRE); and
(v) an adeno-associated virus (AAV) 3′ inverted terminal repeat (ITR).

2. The isolated nucleic acid of claim 1, wherein the PRPF31 polypeptide is a wild-type PRPF31 polypeptide.

3. The isolated nucleic acid of claim 1 or 2, wherein the nucleic acid sequence encoding the PRPF31 polypeptide is or comprises the nucleic acid sequence of SEQ ID NO: 1.

4. The isolated nucleic acid of any one of claims 1-3, wherein the promoter comprises a human cytomegalovirus (CMV) enhancer or portion thereof, a chicken beta-actin (CBA) promoter or portion thereof, a human ubiquitin C (UbC) enhancer or portion thereof, a splice donor, a splice acceptor, or a combination thereof.

5. The isolated nucleic acid of any one of claims 1-4, wherein the promoter is or comprises the nucleic acid sequence of SEQ ID NO: 4.

6. The isolated nucleic acid of any one of claims 1-5, wherein the 5′ ITR is or comprises an AAV2 5′ ITR and/or the 3′ ITR is or comprises an AAV2 3′ ITR.

7. The isolated nucleic acid of any one of claims 1-6, wherein the 5′ ITR is or comprises the nucleic acid sequence of SEQ ID NO: 2.

8. The isolated nucleic acid of any one of claims 1-7, wherein the 3′ ITR is or comprises the nucleic acid sequence of SEQ ID NO: 3.

9. The isolated nucleic acid of claim 8, wherein the WPRE is or comprises the nucleic acid sequence of SEQ ID NO: 5.

10. The isolated nucleic acid of any one of claims 1-9, wherein the isolated nucleic acid further comprises a 3′ untranslated region (UTR) element.

11. The isolated nucleic acid of claim 10, wherein the 3′ UTR element comprises a polyadenylation signal.

12. The isolated nucleic acid of claim 11, wherein the polyadenylation signal comprises a human beta-globin polyadenylation signal.

13. The isolated nucleic acid of claim 11 or 12, wherein the polyadenylation signal is or comprises the nucleic acid sequence of SEQ ID NO: 6.

14. The isolated nucleic acid of any one of claims 1-13, wherein the isolated nucleic acid is or comprises the nucleic acid sequence of SEQ ID NO: 7.

15. A vector comprising the isolated nucleic acid of any one of claims 1-14.

16. The vector of claim 15, wherein the vector is a viral vector.

17. The vector of claim 15, wherein the vector is a recombinant AAV (rAAV) vector.

18. A recombinant adeno-associated virus (rAAV) comprising:
(i) a capsid; and
(ii) the isolated nucleic acid of any one of claims 1-14 or the vector of any one of 15-17.

19. The rAAV of claim 18, wherein the capsid is an AAV2 capsid.

20. A pharmaceutical composition comprising the isolated nucleic acid of any one of claims 1-14, the vector of any one of claims 15-17, or the rAAV of claim 18 or 19.

21. The pharmaceutical composition of claim 20, further comprising a pharmaceutically acceptable carrier or excipient.

22. A cell comprising the isolated nucleic acid of any one of claims 1-14, the vector of any one of claims 15-17, or the rAAV of claim 18 or 19.

23. A method of increasing expression of PRPF31 in a subject, the method comprising administering to the subject a therapeutically effective amount of the isolated nucleic acid of any one of claims 1-14, the vector of any one of claims 15-17, the rAAV of claim 18 or 19, or the pharmaceutical composition of claim 20 or 21.

24. The method of claim 23, wherein the subject is susceptible to or suffering from a disease, disorder, or condition.

25. A method of treating a subject susceptible to or suffering from a disease, disorder, or condition, the method comprising administering to the subject a therapeutically effective amount of the isolated nucleic acid of any one of claims 1-14, the vector of any one of claims 15-17, the rAAV of claim 18 or 19, or the pharmaceutical composition of claim 20 or 21.

26. The method of claim 24 or 25, wherein the disease, disorder, or condition is retinal degeneration.

27. The method of claim 26, wherein the disease, disorder, or condition is retinitis pigmentosa.

28. The method of claim 27, wherein the disease, disorder, or condition is retinitis pigmentosa-11 (RP11).

29. The method of any one of claims 23-28, wherein the subject has a mutation in one or both PRPF31 genes.

30. The method of any one of claims 23-29, wherein the subject is a human.

31. The method of any one of claims 23-30, wherein the isolated nucleic acid, vector, rAAV, or pharmaceutical composition is administered by subretinal injection.

32. The method of any one of claims 23-31, wherein the vector, rAAV, or pharmaceutical composition is administered at a dose within a range of about 10⁸ to about 10¹⁴ vector genome copies (vg) per eye.

33. The method of any one of claims 23-32, wherein the vector, rAAV, or pharmaceutical composition is administered at a dose of about 10⁸, about 10⁹, about 10¹⁰, about 10¹¹, about 10¹², about 10¹³, or about 10¹⁴ vector genome copies (vg) per eye.

34. The method of any one of claims 23-33, wherein the method further comprises administering one or more immunosuppressants.

35. The method of claim 34, wherein one or more immunosuppressants are administered before administration of the isolated nucleic acid, vector, rAAV, or pharmaceutical composition.

36. The method of any one of claims 34-35, wherein one or more immunosuppressants are administered concurrently to administration of the isolated nucleic acid, vector, rAAV, or pharmaceutical composition.

37. The method of any one of claims 34-36, wherein one or more immunosuppressants are administered following administration of the isolated nucleic acid, vector, rAAV, or pharmaceutical composition.

38. The method of any one of claims 34-38, wherein one or more immunosuppressants comprise a steroid.


SEQUENCE LISTING (8 sequences)

SEQ ID NO: 1 - DNA, 1500 bp, Homo sapiens
  source 1..1500: mol_type = genomic DNA
atgtctctggcagatgagctcttagctgatctcgaagaggcagcagaagaggaggaaggaggaagctatggggaggaagaagaggagccagcgatcgaggatgtgcaggaggagacacagctggatctttccggggattcagtcaagaccatcgccaagctatgggatagtaagatgtttgctgagattatgatgaagattgaggagtatatcagcaagcaagccaaagcttcagaagtgatgggaccagtggaggccgcgcctgaataccgcgtcatcgtggatgccaacaacctgaccgtggagatcgaaaacgagctgaacatcatccataagttcatccgggataagtactcaaagagattccctgaactggagtccttggtccccaatgcactggattacatccgcacggtcaaggagctgggcaacagcctggacaagtgcaagaacaatgagaacctgcagcagatcctcaccaatgccaccatcatggtcgtcagcgtcaccgcctccaccacccaggggcagcagctgtcggaggaggagctggagcggctggaggaggcctgcgacatggcgctggagctgaacgcctccaagcaccgcatctacgagtatgtggagtcccggatgtccttcatcgcacccaacctgtccatcattatcggggcatccacggccgccaagatcatgggtgtggccggcggcctgaccaacctctccaagatgcccgcctgcaacatcatgctgctcggggcccagcgcaagacgctgtcgggcttctcgtctacctcagtgctgccccacaccggctacatctaccacagtgacatcgtgcagtccctgccaccggatctgcggcggaaagcggcccggctggtggccgccaagtgcacactggcagcccgtgtggacagtttccacgagagcacagaagggaaggtgggctacgaactgaaggatgagatcgagcgcaaattcgacaagtggcaggagccgccgcctgtgaagcaggtgaagccgctgcctgcgcccctggatggacagcggaagaagcgaggcggccgcaggtaccgcaagatgaaggagcggctggggctgacggagatccggaagcaggccaaccgtatgagcttcggagagatcgaggaggacgcctaccaggaggacctgggattcagcctgggccacctgggcaagtcgggcagtgggcgtgtgcggcagacacaggtaaacgaggccaccaaggccaggatctccaagacgctgcagcggaccctgcagaagcagagcgtcgtatatggcgggaagtccaccatccgcgaccgctcctcgggcacggcctccagcgtggccttcaccccactccagggcctggagattgtgaacccacaggcggcagagaagaaggtggctgaggccaaccagaagtatttctccagcatggctgagttcctcaaggtcaagggcgagaagagtggccttatgtccacctga

SEQ ID NO: 2 - DNA, 130 bp, synthetic construct
  source 1..130: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 3 - DNA, 141 bp, synthetic construct
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 4 - DNA, 1123 bp, synthetic construct
  source 1..1123: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactaaaacaggtaagtccggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgatgcctctactaaccatgttcatgttttctttttttttctacaggtcctgggtgacgaacag

SEQ ID NO: 5 - DNA, 590 bp, Woodchuck hepatitis virus
  source 1..590: mol_type = genomic DNA
caatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 6 - DNA, 395 bp, Homo sapiens
  source 1..395: mol_type = genomic DNA
gctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcatatgccttattcatccctcagaaaaggattcaagtagaggcttgatttggaggttaaagttttgctatgctgtatttta

SEQ ID NO: 7 - DNA, 3895 bp, synthetic construct
  source 1..3895: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactaaaacaggtaagtccggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgatgcctctactaaccatgttcatgttttctttttttttctacaggtcctgggtgacgaacaggccaccatgtctctggcagatgagctcttagctgatctcgaagaggcagcagaagaggaggaaggaggaagctatggggaggaagaagaggagccagcgatcgaggatgtgcaggaggagacacagctggatctttccggggattcagtcaagaccatcgccaagctatgggatagtaagatgtttgctgagattatgatgaagattgaggagtatatcagcaagcaagccaaagcttcagaagtgatgggaccagtggaggccgcgcctgaataccgcgtcatcgtggatgccaacaacctgaccgtggagatcgaaaacgagctgaacatcatccataagttcatccgggataagtactcaaagagattccctgaactggagtccttggtccccaatgcactggattacatccgcacggtcaaggagctgggcaacagcctggacaagtgcaagaacaatgagaacctgcagcagatcctcaccaatgccaccatcatggtcgtcagcgtcaccgcctccaccacccaggggcagcagctgtcggaggaggagctggagcggctggaggaggcctgcgacatggcgctggagctgaacgcctccaagcaccgcatctacgagtatgtggagtcccggatgtccttcatcgcacccaacctgtccatcattatcggggcatccacggccgccaagatcatgggtgtggccggcggcctgaccaacctctccaagatgcccgcctgcaacatcatgctgctcggggcccagcgcaagacgctgtcgggcttctcgtctacctcagtgctgccccacaccggctacatctaccacagtgacatcgtgcagtccctgccaccggatctgcggcggaaagcggcccggctggtggccgccaagtgcacactggcagcccgtgtggacagtttccacgagagcacagaagggaaggtgggctacgaactgaaggatgagatcgagcgcaaattcgacaagtggcaggagccgccgcctgtgaagcaggtgaagccgctgcctgcgcccctggatggacagcggaagaagcgaggcggccgcaggtaccgcaagatgaaggagcggctggggctgacggagatccggaagcaggccaaccgtatgagcttcggagagatcgaggaggacgcctaccaggaggacctgggattcagcctgggccacctgggcaagtcgggcagtgggcgtgtgcggcagacacaggtaaacgaggccaccaaggccaggatctccaagacgctgcagcggaccctgcagaagcagagcgtcgtatatggcgggaagtccaccatccgcgaccgctcctcgggcacggcctccagcgtggccttcaccccactccagggcctggagattgtgaacccacaggcggcagagaagaaggtggctgaggccaaccagaagtatttctccagcatggctgagttcctcaaggtcaagggcgagaagagtggccttatgtccacctgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcatatgccttattcatccctcagaaaaggattcaagtagaggcttgatttggaggttaaagttttgctatgctgtattttaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 8 - protein, 6 aa, synthetic construct
HHHHHH